(19) United States
(12) Patent Application Publication
(10) Pub. No.: US 2025/0366979 A1
(43) Pub. Date: Dec. 4, 2025

(54) DEVICES AND METHODS OF MAKING AND USE THEREOF

(71) Applicant: THE METHODIST HOSPITAL, Houston, TX (US)

(72) Inventor: Aldona SPIEGEL, Houston, TX (US)

(21) Appl. No.: 18/875,400

(22) PCT Filed: Jun. 16, 2023
(86) PCT No.: PCT/US2023/025599

§ 371 Date: Dec. 16, 2024

Related U.S. Application Data
(60) Provisional application No. 63/352,748, filed on Jun. 16, 2022.

(51) Int. Cl. A61F 2/12; A61L 27/26; A61L 27/38; A61L 27/54; A61L 27/56; A61L 27/58; B33Y 50/00; B33Y 80/00; C08L 67/04; C08L 71/02; C08L 71/08

(52) U.S. Cl. CPC A61F 2/12; A61L 27/26; A61L 27/3808; A61L 27/3839; A61L 27/54; A61L 27/56; A61L 27/58; B33Y 50/00; B33Y 80/00; C08L 67/04; C08L 71/02; C08L 71/08; A61L 2300/416; A61L 2430/34

(57) ABSTRACT

Disclosed herein are devices and methods of making and use thereof.

### CROSS-REFERENCE TO RELATED APPLICATIONS

This application claims the benefit of priority to U.S. Provisional Application No. 63/352,748 filed Jun. 16, 2022, which is hereby incorporated herein by reference in its entirety.

### BACKGROUND

Breast cancer is a multifaceted disease. The treatment of which goes far beyond the initial declaration of “Cancer-Free.” Many breast cancer patients have a mastectomy or lumpectomy as part of their cancer treatment. These women often experience significant anxiety and depression regarding their mastectomy or lumpectomy. Breast reconstruction can reduce anxiety and depression in these patients. Reconstruction, as it stands, is an imperfect science. Currently, implants made out of silicone are traditionally used for breast enhancement, but recently received a black box warning by the FDA, due to risk of implant failure, a rare form of ALCL that is associated with some textured implants, and breast implant illness. The silicone implant technology has not advanced substantially over the past three decades. The current alternative is autologous reconstruction, which permits restoration of the breast with adipose tissue from a donor site, by microsurgically connecting the blood supply. However, this is a more complex operation, requires longer recovery and not every patient has excess tissue available for this purpose.

Similar limitations are present for reconstruction and/or enhancement of other anatomical locations.

With current technologies and techniques needing improvement, new methods of reconstruction and/or enhancement are necessary. The devices and methods discussed herein address these and other needs.

### SUMMARY

In accordance with the purposes of the disclosed devices and methods as embodied and broadly described herein, the disclosed subject matter relates to devices and methods of making and use thereof.

Additional advantages of the disclosed devices and methods will be set forth in part in the description which follows, and in part will be obvious from the description. The advantages of the disclosed devices and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. BioBreast Model 1-free form curve.

FIG. 2. BioBreast Model 1-free form curve.

FIG. 3. BioBreast Model 2-Fibonacci.

FIG. 4. BioBreast Model 2-Fibonacci.

FIG. 5. BioBreast Model 2-Fibonacci.

FIG. 6. BioBreast Model 2-Fibonacci.

FIG. 7. BioBreast Model 2-Fibonacci.

FIG. 8. BioBreast Model 2-Fibonacci.

FIG. 9. BioBreast Model 2-Fibonacci.

FIG. 10. BioBreast Model 2-Fibonacci.

FIG. 11. BioBreast Model 2-Fibonacci.

FIG. 12. BioBreast Model 2-Fibonacci.

FIG. 13. BioBreast Model 3-Combination.

FIG. 14. BioBreast Model 3-Combination.

FIG. 15. BioBreast Model 3-Combination.

FIG. 16. BioBreast-Internal Matrix.

FIG. 17. BioBreast-Internal Matrix

FIG. 18. BioBreast-Internal Matrix

FIG. 19. BioBreast-Internal Matrix

FIG. 20. BioBreast-Internal Matrix

FIG. 21. BioBreast-linearly oriented, larger diameter channels.

FIG. 22. Examples of different phyllotaxis patterns.

FIG. 23. An example phyllotactic spiral.

FIG. 24. Antero posterior projections design.

FIG. 25. Fibonacci sequence.

FIG. 26. Schematic view of an example device as disclosed herein according to one implementation.

FIG. 27. Another schematic view of the example device shown in FIG. 26 as disclosed herein according to one implementation.

FIG. 28. Another schematic view of the example device shown in FIG. 26-FIG. 27 as disclosed herein according to one implementation.

FIG. 29. Another schematic view of the example device shown in FIG. 26-FIG. 28 as disclosed herein according to one implementation.

FIG. 30. Another schematic view of the example device shown in FIG. 26-FIG. 29 as disclosed herein according to one implementation.

FIG. 31. Photograph of an example device as disclosed herein according to one implementation.

FIG. 32. Photograph of an example device as disclosed herein according to one implementation.

FIG. 33. Viscosity assessment as a function of temperature.

FIG. 34. Photograph of an example device fabricated using Pluronic at 25% w/v concentration, which becomes a stiff gel at room temperature.

FIG. 35. Stress vs. Strain for non-sterilized devices.

FIG. 36. Stress vs. Strain for devices sterilized using EtO.

FIG. 37. Youngs Modulus of sterilized and non-sterilized devices.

FIG. 38. MicroCT image of an example device.

FIG. 39. Image showing measurements of internal diameters of tubes of an example device.

FIG. 40. Image showing measurements of the empty surface of the hollow scaffolds of an example device.

FIG. 41. Photograph of rat with surgical markings.

FIG. 42. Photograph of anesthetized rat with surgical markings before incision.

FIG. 43. Photograph of anesthetized rat with surgical markings during incision.

FIG. 44. Photograph of anesthetized rat with surgical markings after incision.

FIG. 45. Photograph of fat harvesting from rat.

FIG. 46. Photograph of fat harvesting from rat.

FIG. 47. Photograph of pocket dissection.

FIG. 48. Photograph of pocket dissection.

FIG. 49. Photograph of filling construct with processed fat.

FIG. 50. Photograph of filling construct with processed fat.

FIG. 51. Photograph of construct filled with processed fat.

FIG. 52. Photograph filled construct, with inlet to be removed after filling indicated by dotted outline.

FIG. 53. Schematic view of an example device as disclosed herein according to one implementation, after inlet removal.

FIG. 54. Another schematic view of the example device shown in FIG. 53 as disclosed herein according to one implementation.

FIG. 55. Another schematic view of the example device shown in FIG. 53-FIG. 54 as disclosed herein according to one implementation.

FIG. 56. Photograph showing surgical device placement.

FIG. 57. Photograph showing surgical device placement.

FIG. 58. Photograph of closed surgical incision after device placement.

FIG. 59. MRI 7T image from rat cadaver with the implanted device.

FIG. 60. MRI 7T image from rat cadaver with the implanted device.

FIG. 61. MRI 9.4T image of empty device (right) and fat filled device (left) embedded in agarose gel phantom.

FIG. 62. Photograph of rat A-1 post-surgery.

FIG. 63. 7T MRI image of rat A-1 two weeks post-surgery.

FIG. 64. 7T MRI image of rat A-1 two weeks post-surgery.

FIG. 65. Photograph of rat A-2 post-surgery.

FIG. 66. Photograph of rat A-2 post-surgery.

FIG. 67. 7T MRI image of rat A-2 two weeks post-surgery (endpoint for rat A-2).

FIG. 68. 7T MRI image of rat A-2 two weeks post-surgery (endpoint for rat A-2).

FIG. 69. 9.4T MRI image of left side (fat-filled) device in rat A-2 two weeks post-surgery (endpoint for rat A-2).

FIG. 70. 9.4T MRI image of right side (empty) device in rat A-2 two weeks post-surgery (endpoint for rat A-2).

FIG. 71. 9.4T MRI image of rat A-2 two weeks post-surgery (endpoint for rat A-2).

FIG. 72. MicroCT angiography of device from rat A-2 two weeks post-surgery (endpoint for rat A-2).

FIG. 73. Illustration of device before implantation.

FIG. 74. Illustration of differences between the microCT image from rat A-2 (FIG. 72) after implantation with the microCT-image of the device before implantation (FIG. 38).

FIG. 75. Photograph of rat A-3 post-surgery.

FIG. 76. Photograph of rat A-3 post-surgery.

FIG. 77. 7T MRI image of rat A-3 two weeks post-surgery.

FIG. 78. 7T MRI image of rat A-3 two weeks post-surgery.

FIG. 79. Photograph of rat A-4 post-surgery.

FIG. 80. Photograph of rat A-4 post-surgery.

FIG. 81. 7T MRI image of rat A-4 two weeks post-surgery.

FIG. 82. 7T MRI image of rat A-4 two weeks post-surgery.

FIG. 83. Schematic view of an example device as disclosed herein according to one implementation.

FIG. 84. Schematic view of an example device as disclosed herein according to one implementation.

FIG. 85. Schematic view of an example device as disclosed herein according to one implementation.

FIG. 86. Schematic view of an example device as disclosed herein according to one implementation.

FIG. 87. Schematic view of an example device as disclosed herein according to one implementation.

### DETAILED DESCRIPTION

The devices and methods described herein may be understood more readily by reference to the following detailed description of specific aspects of the disclosed subject matter and the Examples included therein.

Before the present devices and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof”′ as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Biocompatible” and “biologically compatible”, as used herein, generally refer to compounds and/or compositions that are, along with any metabolites or degradation products thereof, generally non-toxic to normal cells and tissues, and which do not cause any significant adverse effects to normal cells and tissues when cells and tissues are incubated (e.g., cultured) in their presence.

The term “biodegradable” or “bioresorbable” as used herein refers to a material or substance wherein physical dissolution and/or chemical degradation is effected under physiological conditions.

As used herein, a “chamber” generally refers to a volume that is at least partially enclosed, and in some instances fully enclosed. A chamber can, for example, be hollow. In some examples, a chamber can be at least partially filled with a substance.

“Continuous,” as used herein, generally refers to a phase such that all points within the phase are directly connected three-dimensionally, so that for any two points within a continuous phase, there exists a path in three-dimensional space which connects the two points without leaving the phase.

As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

As used herein, “reduce” or other forms of the word, such as “reducing” or “reduction,” refers to lowering of an event or characteristic (e.g., microbe population/infection). It is understood that the reduction is typically in relation to some standard or expected value. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “molecular weight” refers to the number average molecular weight as measured by ¹H NMR spectroscopy, unless indicated otherwise.

### Devices

Disclosed herein are devices configured to be inserted into an anatomical location of a subject.

In some examples, the devices can comprise a bioresorbable polymer; wherein, when the device is inserted into the anatomical location of the subject, then device becomes encapsulated by vascular tissue and the bioresorbable polymer is resorbed over an amount of time, thereby forming a chamber in place of the bioresorbable polymer; wherein the chamber is defined by a boundary formed from the vascular tissue; wherein the chamber is perfusable; such that chamber is configured to be at least partially filled with a first plurality of cells. In some examples, the chamber can be filled with a combination of different types of cells, allowing for versatility in the types of cells used.

For example, the devices when inserted into the anatomical location of the subject, involves the creation of smaller chambers using a bioresorbable polymer that can support the growth of cells depending on their size and proximity to blood flow. In some examples, the bioresorbable polymer can be resorbed quickly, since vessel ingrowth can occur within the first 24 hours after the device has been inserted into the anatomical location. The devices and methods disclosed herein can support cell growth and an functionality within the vascularized chamber. In some examples, the boundary formed by the vascular tissue can further comprise an inner lining or matrix that can support the growth and/or organization of cells within the chamber.

In some examples, the device, before the bioresorbable polymer is resorbed, further includes a compartment defined by the bioresorbable polymer, wherein the compartment is configured to be at least partially filled with a second plurality of cells before or shortly after being inserted into the anatomical location of the subject. In some examples, the compartment is non-filled (e.g., empty) before and during insertion into the anatomical location of the subject. In some examples, the compartment is at least partially filled with the second plurality of cells before the bioresorbable polymer is resorbed (e.g., before or shortly after being inserted into the anatomical location of the subject). In some examples, the compartment is at least partially filled with the second plurality of cells after the bioresorbable polymer is resorbed. For example, the device is configured to be filled or refilled at a later stage.

In some examples, the chamber is at least partially filled with the first plurality of cells after the bioresorbable polymer is resorbed

In some examples, the first plurality of cells and/or the second plurality of cells independently comprise adipose tissue. In some examples, the first plurality of cells and/or the second plurality of cells can independently be genetically modified or manipulated to enhance their regenerative potential. In some examples, the first plurality of cells and/or the second plurality of cells can independently comprise stem cells or progenitor cells capable of differentiating into adipose tissue or other desired cell types.

In some examples, the devices comprises a plurality of conduits comprising the bioresorbable polymer; wherein, when the device is inserted into the anatomical location of the subject, then device becomes encapsulated by vascular tissue and the bioresorbable polymer is resorbed over an amount of time, thereby forming a plurality of channels in place of the plurality of conduits; wherein each channel is defined by a boundary formed from the vascular tissue; wherein the plurality of channels are perfusable; and wherein each of the plurality of channels is configured to receive and be at least partially filled cells, such as adipose tissue. In some examples, the device is non-filled (e.g., empty) before and during insertion into the anatomical location of the subject. For example, the device can be non-filled (e.g., empty) before and during insertion into the anatomical location of the subject and can subsequently be at least partially filled with the cells after insertion. In some examples, the device can be at least partially filled with the cells before or after insertion into the anatomical location. The cells can, for example, be delivered via a port or directly injected into the device.

In some examples, the devices comprises a plug comprising a bioresorbable polymer; and a plurality of conduits comprising the bioresorbable polymer; wherein, when the device is inserted into the anatomical location of the subject, then device becomes encapsulated by vascular tissue and the bioresorbable polymer is resorbed over an amount of time, thereby forming a plurality of channels in place of the plurality of conduits and a port in place of the plug; wherein each channel is defined by a boundary formed from the vascular tissue; wherein the port is further defined by the vascular tissue; wherein the port and the plurality of channels are perfusable; and wherein each of the plurality of channels is fluidly connected to the port; such that port is configured to receive cells, such as adipose tissue, and the plurality of channels are configured to be at least partially filled with cells, such as adipose tissue, delivered through the port. In some examples, the device is non-filled (e.g., empty) before and during insertion into the anatomical location of the subject.

In some examples, the plurality of conduits are solid, such that boundary defining each channel is an outer wall.

In some examples, the plurality of conduits are hollow, such that the plurality of conduits comprise a wall defining a lumen, the wall comprising the bioresorbable polymer, and each channel is defined by an inner wall and an outer wall, the inner wall and the outer wall being vascular tissue.

In some examples, the plurality of conduits comprises: a first population of conduits; and a second population of conduits; each of the first population and the second population including one or more of the plurality of conduits; such that the plurality of channels comprises a first population of channels and a second population of channels, each of the first population of channels and the second population of channels including one or more of the plurality of channels; wherein the first population of conduits are solid, such that boundary defining each of the first population of channels is an outer wall; and wherein the second population of conduits are hollow such that the each of the second population of conduits comprises a wall defining a lumen, the wall comprising the bioresorbable polymer, and each of the second population of channels is defined by an inner wall and an outer wall, the inner wall and the outer wall being vascular tissue.

In some examples, each of the plurality of channels, the first population of channels, the second population of channels, or a combination thereof independently can be at least partially filled with cells, such as adipose tissue, after the bioresorbable polymer is resorbed.

Also disclosed herein are devices comprising: a first plurality of conduits, wherein each of the first plurality of conduits comprises a wall defining a lumen, the wall comprising the bioresorbable polymer; wherein the lumen of each of the first plurality of conduits is perfusable; wherein the lumen of each of the first plurality of conduits is configured to be at least partially filled with a first portion of cells, such as adipose tissue; wherein the device is configured to be at least partially filled with the first portion of cells, such as adipose tissue, before or shortly after being inserted into the anatomical location of the subject (e.g., before the bioresorbable polymer is resorbed). In some examples, the device is non-filled (e.g., empty) before and during insertion into the anatomical location of the subject. For example, the device can be non-filled (e.g., empty) before and during insertion into the anatomical location of the subject and can subsequently be at least partially filled with the first portion of cells after insertion. In some examples, the device can be at least partially filled with the first portion of cells before or after insertion into the anatomical location. The first portion of cells can, for example, be delivered via a port or directly injected into the device. When the device is inserted into the anatomical location of the subject, then device becomes encapsulated by vascular tissue and the bioresorbable polymer is resorbed, thereby forming a first plurality of channels in place of the first plurality of conduits, each of the first plurality of channels being defined by a boundary formed from the vascular tissue and each of the first plurality of channels being at least partially filled with the first portion of cells, such as adipose tissue. In some examples, the cells can be a combination of different types of cells, allowing for versatility in the types of cells used.

Also disclosed herein are devices comprising: a port defined by a boundary comprising a bioresorbable polymer; and a first plurality of conduits, wherein each of the first plurality of conduits comprises a wall defining a lumen, the wall comprising the bioresorbable polymer; wherein the port and the lumen of each of the first plurality of conduits is perfusable; wherein the lumen of each of the first plurality of conduits is fluidly connected to the port; such that the port is configured to receive a first portion of cells, such as adipose tissue, and the lumen of each of the first plurality of conduits is configured to be at least partially filled with the first portion of cells, such as adipose tissue, delivered through the port; wherein the device is configured to be at least partially filled with the first portion of cells, such as adipose tissue, before or shortly after being inserted into the anatomical location of the subject (e.g., before the bioresorbable polymer is resorbed). In some examples, the device is non-filled (e.g., empty) before and during insertion into the anatomical location of the subject. When the device is inserted into the anatomical location of the subject, then device becomes encapsulated by vascular tissue and the bioresorbable polymer is resorbed, thereby forming a first plurality of channels in place of the first plurality of conduits, each of the first plurality of channels being defined by a boundary formed from the vascular tissue and each of the first plurality of channels being at least partially filled with the first portion of cells, such as adipose tissue. In some examples, the cells can be a combination of different types of cells, allowing for versatility in the types of cells used.

For example, the devices when inserted into the anatomical location of the subject, involves the creation of smaller chambers using a bioresorbable polymer that can support the growth of cells depending on their size and proximity to blood flow. In some examples, the bioresorbable polymer can be resorbed quickly, since vessel ingrowth can occur within the first 24 hours after the device has been inserted into the anatomical location. The devices and methods disclosed herein can support cell growth and an functionality within the vascularized chamber.

In some examples, the device, after the bioresorbable polymer is resorbed, is further configured to receive a second portion of cells, such as adipose tissue, delivered through the port (when present) or directly to the first plurality of channels, such that the first plurality of channels are further configured be at least partially filled with a second portion of cells, such as adipose tissue.

In some examples, the device further comprises: a second plurality of conduits comprising a second bioresorbable polymer, the second plurality of conduits being solid; such that, when the device is inserted into the anatomical location of the subject, then the second plurality of conduits becomes encapsulated by vascular tissue and the second bioresorbable polymer is resorbed over a second amount of time, thereby forming a second plurality of channels in place of the second plurality of conduits; wherein each of the second plurality of channels is defined by a boundary formed from the vascular tissue; wherein the second plurality of channels are perfusable; and wherein each of the second plurality of channels are fluidly connected to the port (when present); such that the port (when present) is configured to receive a third portion of cells, such as adipose tissue, and second the plurality of channels are configured to be at least partially filled with the third portion of cells, such as adipose tissue delivered directly thereto or through the port (when present). The second bioresorbable polymer can be the same as or different than the bioresorbable polymer. The second amount of time can be the same as or different than the amount of time. The third portion of cells (e.g. adipose tissue) can be the same as or different than second portion of cells (e.g., adipose tissue).

Each of the plurality of conduits each of the first plurality of conduits, each of the second plurality of conduits, or a combination thereof independently can, for example, extend from a proximal end to a distal end opposite and spaced apart from the proximal end along a longitudinal axis. In some examples, each of the plurality of conduits, each of the first plurality of conduits, each of the second plurality of conduits, or a combination thereof independently can further comprise one or more lobes or branches extending from the longitudinal axis.

In some examples, each of the plurality of conduits, each of the first plurality of conduits, each of the second plurality of conduits, or a combination thereof can independently have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape can be substantially circular, ovate, ovoid, elliptic, triangular, rectangular, polygonal, etc. In some examples, the cross-sectional shape can be substantially circular. In some examples, the cross-sectional shape can vary along the longitudinal axis.

Each of the plurality of conduits, each of the first plurality of conduits, each of the second plurality of conduits, or a combination thereof can independently have an average characteristic dimension. The term “characteristic dimension,” as used herein, refers to the largest straight-line distance between two points in the plane of the cross-sectional shape. “Average characteristic dimension” and “mean characteristic dimension” are used interchangeably herein, and generally refer to the statistical mean characteristic dimension. For example, when the cross-sectional shape is substantially circular and the average characteristic dimension can refer to the average diameter. In some examples, the average characteristic dimension of each of the plurality of conduits, each of the first plurality of conduits, each of the second plurality of conduits, or a combination thereof independently can vary along the longitudinal axis (e.g., tapered, stepped, etc.).

In some examples, the device is configured to maximize the volume of cells (e.g., adipose tissue) while ensuring that the average maximum distance between the cells (e.g., adipose tissue) within any part of the device and the vascular tissue is such that the cells (e.g., adipose tissue) is/are within an adequate proximity to the vascular tissue, and thus within an adequate proximity to a blood supply, to nourish the cells (e.g., adipose tissue) and ensure that the cells (e.g., adipose tissue) remains viable.

In some examples, the chamber, the compartment, each of the plurality of conduits, each of the first plurality of conduits, each of the second plurality of conduits, or a combination thereof independently is/are further lined with a third plurality of cells, such as endothelial cells.

In some examples, the devices can be filled with a combination of different types of cells, allowing for versatility in the types of cells used. For example, the chamber, the compartment, each of the plurality of conduits, each of the first plurality of conduits, each of the plurality of channels, each of the first population of channels, each of the second population of channels, each of the first plurality of channels, each of the second plurality of channels, or a combination thereof are independently configured to be at least partially filled with one or more types of cells, such as a combination of different types of cells, allowing for versatility in the types of cells used.

In some examples, the devices can be non-filled (e.g., empty), partially filled, or fully filled before and during insertion into the anatomical location of the subject. In some examples, the devices can be non-filled (e.g., empty) before and during insertion into the anatomical location of the subject, and the devices can subsequently be partially or fully filled after insertion.

In some examples, the device is at least partially filled with a nutrient medium, for example to support cell growth and functionality.

The devices are independently configured to be at least partially filled with cells, such as adipose tissue. In some examples, the cells can be genetically modified or manipulated to enhance their regenerative potential. In some examples, the cells can comprise stem cells or progenitor cells capable of differentiating into adipose tissue or other desired cell types.

For example, the chamber, the compartment, each of the plurality of conduits, each of the first plurality of conduits, each of the plurality of channels, each of the first population of channels, each of the second population of channels, each of the first plurality of channels, each of the second plurality of channels, or a combination thereof are independently configured to be at least partially filled with cells, such as adipose tissue. In some examples, the chamber, the compartment, each of the plurality of conduits, each of the first plurality of conduits, each of the plurality of channels, each of the first population of channels, each of the second population of channels, each of the first plurality of channels, each of the second plurality of channels, or a combination thereof are at least partially filled with cells, such as adipose tissue. In some examples, the chamber, the compartment, each of the plurality of conduits, each of the first plurality of conduits, each of the plurality of channels, each of the first population of channels, each of the second population of channels, each of the first plurality of channels, each of the second plurality of channels, or a combination thereof are at least partially filled with a mixture comprising adipose tissue. The mixture can, for example, further comprise an additional component, which can, for example, improve the uptake of the fat. For example, the additional component can comprise platelets, plasma, platelet-rich plasma (PRP), stem cells, a protein, or a combination thereof. In some examples, the adipose tissue comprises autologous adipose tissue.

In some examples, the device is formed from a model based on a tessellation of polyhedrons. For example, the device can formed from a computational 3D space-filling model.

In some examples, the device has a three-dimensional parametric teardrop shape. In some examples, the device has a three-dimensional parametric teardrop shape following the Fibonacci equation.

In some examples, the device can be anatomically designed for the subject.

In some examples, the anatomical location comprises at least a portion of a breast of the subject. In some examples, the anatomical location comprises a breast of the subject.

The bioresorbable polymer and/or the second bioresorbable polymer (when present) can independently comprise any suitable material. For example, the bioresorbable polymer and/or the second bioresorbable polymer (when present) can independently comprise poly(ethylene glycol) diacrylate (PEGDA), poly(ethylene glycol) dimethacrylate (PEGDMA), poly(ethylene glycol) diacrylamide (PEGDAAm), gelatin methacrylate (GelMA), collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, Chitosan methacrylate, polyethylene glycol norbornene, polyethylene glycol dithiol, thiolated gelatin, thiolated chitosan, thiolated silk, PEG based peptide conjugates, cell-adhesive poly(ethylene glycol), MMP-sensitive poly(ethylene glycol), PEGylated fibrinogen, or a combination thereof. The second bioresorbable polymer can be the same as or different than the bioresorbable polymer

In some examples, the bioresorbable polymer and/or the second bioresorbable polymer (when present) can each independently comprise a polyester, such as poly(glycerol-dodecanoate) (PGD).

In some examples, the bioresorbable polymer and/or the second bioresorbable polymer (when present) can each independently comprise a poly(ether-ester). In some examples, the bioresorbable polymer and/or the second bioresorbable polymer (when present) can each independently comprise polydioxanone (PDO).

In some examples, the bioresorbable polymer and/or the second bioresorbable polymer (when present) can each independently comprise a polyolefin, such as polypropylene.

In some examples, the bioresorbable polymer and/or the second bioresorbable polymer (when present) can each independently comprise polyglycolic acid (PGA).

In some examples, the bioresorbable polymer and/or the second bioresorbable polymer (when present) can each independently comprise polyglycolic acid (PGA) or a copolymer thereof, polylactic acid (PLA) or a copolymer thereof, polycaprolactone (PCL) or a copolymer thereof, or a combination thereof.

In some examples, the bioresorbable polymer and/or the second bioresorbable polymer (when present) can each independently comprise a bioink. Examples of bioinks include, but are not limited to, alginate-based bioinks, gelatin-based bioinks (e.g., GelMA-Gelatin Methacryloyl), collagen-based bioinks (e.g., Type I collagen, Type II collagen), fibrin-based bioinks, chitosan-based bioinks, hyaluronic acid-based bioinks, Matrigel-based bioinks, silk fibroin-based bioinks, cellulose-based bioinks, polyethylene glycol-based bioinks (PEG-based bioinks), polycaprolactone-based bioinks (PCL-based bioinks), poly(lactic-co-glycolic acid)-based bioinks (PLGA-based bioinks), polyvinyl alcohol-based bioinks (PVA-based bioinks), polyurethane-based bioinks, polydopamine-based bioinks, polypeptide-based bioinks, agarose-based bioinks, carboxymethyl cellulose-based bioinks, sodium alginate-gelatin-based bioinks, polyethylene oxide-based bioinks (PEO-based bioinks), and combinations thereof.

In some examples, the bioresorbable polymer and/or the second bioresorbable polymer (when present) independently can be porous.

In some examples, the device can further comprise collagen, for example to provide structural support to the bioresorbable polymer.

In some examples, the device can further comprise a scaffold, such as a porous scaffold.

In some examples, the device can have a compressibility, elasticity, and/or porosity which can be selected to improve fat uptake by the device and/or comfort for the subject. In some examples, the bioresorbable polymer and/or the second bioresorbable polymer (when present) can independently be selected in view of the desired compressibility, elasticity, and/or porosity of the device. In some examples, characteristics of the bioresorbable polymer can be selected to control the speed of resorption to optimize vascularization and perfusion of cells, but also on the macroscopic aspect of the way the device feels for the patient with maximum elasticity and compressibility for the comfort of the patient.

In some examples, the device further comprises a therapeutic agent dispersed within the bioresorbable polymer and/or the second bioresorbable polymer. In some examples, the therapeutic agent is dispersed inhomogeneously throughout the bioresorbable polymer and/or the second bioresorbable polymer (e.g., randomly, along a concentration gradient, etc.). In some examples, the therapeutic agent is dispersed substantially homogeneously throughout the bioresorbable polymer and/or the second bioresorbable polymer.

The therapeutic agent can, for example, comprise an anticancer agent, anti-inflammatory agent, analgesic agent, antimicrobial agent, or a combination thereof. As used herein, antimicrobials include, for example, antibacterials, antifungals, and antivirals.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper (I) cyanide, copper (II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2 (5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl) quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, ZMapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine, and combinations thereof.

In some examples, the therapeutic agent can comprise an anticancer agent. In some examples, the therapeutic agent comprises a chemotherapeutic agent, an immunotherapeutic agent, or a combination thereof.

In some examples, the therapeutic agent can comprise a chemotherapeutic agent. Chemotherapy is the treatment of cancer with one or more cytotoxic anti-neoplastic drugs (e.g., chemotherapeutic agents) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapeutic agents include, but are not limited to, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine,-Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, Oprozomib, and combinations thereof. The anticancer agent can also include biopharmaceuticals such as, for example, antibodies.

Examples of suitable immunotherapeutic agents include, but are not limited to, alemtuzumab, cetuximab (ERBITUX), gemtuzumab, iodine 131 tositumomab, rituximab, trastuzamab (HERCEPTIN), and combinations thereof.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent can comprise an analgesic. Examples of analgesics include, but are not limited to, 1-Iodomorphine; 3-Hydroxymorphinan; 4-Methylpregabalin; A-366,833; ABT-202; Aceburic acid; Acefurtiamine; Acetaminosalol; Acetyldihydrocodeine; Acetylmethadol; Adrenorphin; Alazocine; Algifen; Alimadol; Alletorphine; Alphacetylmethadol; Alphamethadol; Amidorphin; Aminophenazone; Ampyrone; Amrutanjan (balm); Anacin; Anadin; Analgecine; Anazocine; Anileridine; Anilopam; Anodyne; Askit Powders; Aspergum; Aspirin; Axomadol; AZD0328; BC Powder; Befiradol; Benorilate; Betacetylmethadol; Betahydroxyfentanyl; Betamethadol; Bicifadine; Biphalin; Brorphine; Bucetin; Bucinnazine; Butalbital; Butinazocine; Butonitazine; Butorphanol; Cannabidiol; Carbazocine; Cebranopadol; Chlorodyne; Chlorproethazine; Cinchophen; Cogazocine; Conolidine; Conorfone; CR-4056; CR665; Dasolampanel; Deltorphin; Deltorphin I; DepoDur; Desmetramadol; Desomorphine; Dezocine; Diacetylnalorphine; Dichloralphenazone; Difenamizole; Dimenoxadol; Dimepheptanol; Dimethylheptylpyran; Dinalbuphine sebacate; Dipipanone; Diproqualone; Dipyrocetyl; Dosulepin; DSP-2230; Embutramide; Enkephalinase nhibitor; Epibatidine; Epiboxidine; Eptazocine; Esterom; Etazocine; Ethylketazocine; Etodesnitazene; Etonitazepipne; Etonitazepyne; Etorphine; Famotidine; Faxeladol; Fedotozine; Fentanyl; Filenadol; Flumexadol; Flumizole; Fluproquazone; Frakefamide; Funapide; Gabapentin; Gabapentin enacarbil; Gabapentinoid; Glafenine; Homofentanyl; Homprenorphine; Ibazocine; Ibuprofen; Incarvillateine; Indantadol; Isomethadone; Isotonitazene; Isovaline; Kavalactone; Kelatorphan; Ketamine; Ketobemidone; Ketorfanol; Ketorolac; Lactucarium; Leconotide; Levallorphan; Levomepromazine; Levomethadone; Lufuradom; Magnesium alicylate; Mavatrep; Meconopsis horridula; Menabitan; Menthol; Menthoxypropanediol; Meprobamate; Meseclazone; Metacetamol; Metamizole; Methoxyflurane; Metkefamide; Metodesnitazene; Metonitazene; Mexiletine; Migraleve; Mirogabalin; Mitragyna speciosa; Moffett's solution; Moramide intermediate; Morpheridine; Morphiceptin; Morphine; Moxazocine; MP-2001; N-2′-Indolylnaltrexamine; Nabilone; Nafoxadol; Nalbuphine; Nalmexone; Naproxen; Nefopam; Nexeridine; NFEPP; Nimesulide; Noracymethadol; Norlevorphanol; Normethadone; Norpipanone; NS-11394; Oliceridine; Opiorphin; Opiranserin; Opium; Otenaproxesul; Oxilorphan; Paracetamol; Pethidine; PF-05089771; Phenacetin; Phenazone; derivatives thereof; and combinations thereof.

In some examples, the therapeutic agent can comprise an analgesic, such as an opioid. Examples of opioids include, but are not limited to, (α/β)-Meprodine; (α/β)-Prodine; 1-(4-Nitrophenylethyl) piperidylidene-2-(4-chlorophenyl) sulfonamide (W-18); 14-Cinnamoyloxycodeinone; 14-Ethoxymetopon; 14-Hydroxydihydrocodeine; 14-Hydroxymorphine; 14-Methoxymetopon; 14-Phenylpropoxymetopon; 18,19-Dehydrobuprenorphine (HS-599); 18-Methoxycoronaridine; 1-Bromocodeine; 1-Chlorocodeine; 1-Iodomorphine Codeine-6-glucuronide; 1-Nitrocodeine; 2,4-Dinitrophenylmorphine; 3-(3-Methoxyphenyl)-3-ethoxycarbonyltropane; 3-(dimethylamino)-2,2-dimethyl-1-phenylpropan-1-one; 3,14-Diacetyloxymorphone; 3,6-Dibutanoylmorphine; 3-Acetyloxymorphone; 3-Allylfentanyl; 3-Hydroxymorphinan; 3-Methylfentanyl; 3-Methylthiofentanyl; 3-Monoacetylmorphine; 4-Chlorophenylpyridomorphinan; 4-Fluoropethidine; 4-Phenylfentanyl; 5,6-Dihydronorsalutaridine; 5,9 alpha-diethyl-2-hydroxybenzomorphan (5,9-DEHB); 6-Acetyldihydromorphine; 6-Keto Nalbuphine; 6-Methyldihydromorphine; 6-Methylenedihydrodesoxymorphine; 6-Monoacetylcodeine; 6-Monoacetylmorphine; 6-Nicotinoyldihydromorphine; 7-Acetoxymitragynine; 7-Hydroxymitragynine; 7-PET; 7-Spiroindanyloxymorphone; 8,14-Dihydroxydihydromorphinone; 8-Carboxamidocyclazocine (8-CAC); Acetorphine; Acetoxyketobemidone; Acetylcodone; Acetyldihydrocodeine; Acetylmethadol; Acetylmorphone; Acetylpropionylmorphine; AD-1211; ADL-5859; AH-7921; Aknadinine; Akuammidine; Akuammine; Alazocine; Alfentanil; Alimadol; Alletorphine (N-allyl-noretorphine); Allylnorpethidine; Allylprodine; Alphaacetylmethadol; Alphamethadol; Alvimopan; Amentoflavone; Anazocine; Anileridine; Anilopam +HCl; Asimadoline; Axomadol; Azaprocin; AZD-2327; Azidomorphine; BDPC; Benzethidine; Benzhydrocodone; Benzylfentanyl; Benzylmorphine; Betacetylmethadol; Betamethadol; Bezitramide; Bisnortilidine; Bremazocine; Brifentanil; BRL-52537; Bromadol; Bromadoline; Bromocodide; Bromoisopropropyldihydromorphinone; Bromomorphide; BU-48; Buprenorphine; Buprenorphine-3-glucuronide; Butinazocine; Butorphanol; Butyrfentanyl; BW373U86; Carbazocine; Carfentanil; Carperidine; Cephakicine; Cephasamine; Chlornaltrexamine; Chlorodihydrocodide; Chloromorphide; Chloroxymorphamine; Ciprefadol; Ciramadol; Clonitazene; Codeine; Codeine methylbromide; Codeine-N-oxide; Codeine-N-oxide (genocodeine); Codeinone; Codide; Codoxime; Cogazocine; Conorfone (codorphone); Coronaridine; Cyclazocine; Cyclorphan; Cyprenorphine; Cyprodime; Cyproterone acetate; Desmethylclozapine; Desmethylmoramide; Desmethylprodine (MPPP); Desocodeine Desomorphine (dihydrodesoxymorphine); Dextromethadone; Dextromoramide; Dextropropoxyphene (propoxyphene); Dezocine; Diacetyldihydromorphine (dihydroheroin, acetylmorphinol); Diampromide; Dibenzoylmorphine; Dibutyrylmorphine; Diethylthiambutene; Difenoxin; Diformylmorphine; Dihydrocodeine; Dihydrocodeine; Dihydrodesoxycodeine (desocodeine); Dihydroetorphine; Dihydroisocodeine; Dihydromorphine; Dimenoxadol; Dimepheptanol (racemethadol); Dimethylmorphine (6-O-Methylcodeine); Dimethylthiambutene; Dioxaphetyl butyrate; Diphenoxylate; Dipipanone; Dipropanoylmorphine; Doxpicomine; DPI-221; DPI-287; DPI-3290; Drotebanol; Droxypropine; Embutramide; Enadoline; Eptazocine; Eseroline; Etazocine; Ethoheptazine; Ethyldihydromorphine; Ethylketazocine; Ethylmethylthiambutene; Ethylmorphine (dionine); Etonitazene; Etorphine; Etoxeridine (carbetidine); Faxeladol; FE 200665; Fedotozine; Fenfangjine G; Fentanyl; Fluorophen; Furethidine; Gemazocine; GR-89696; Herkinorin; Heroin (diacetylmorphine); Heroin-7,8-oxide; Heterocodeine; Hodgkinsine; Homprenorphine; Hydrocodone; Hydromorphinol; Hydromorphone; Hydroxycodeine; Hydroxypethidine (bemidone); HZ-2; Ibazocine; IBNtxA; Ibogaine; IC-26; ICI-199,441; ICI-204,448; Isoaminile; Isocodeine; Isomethadol; Isomethadone; Isotonitazene; Ketamine; Ketazocine; Ketobemidone; Ketorfanol; KNT-42; Kolokol-1; Lefetamine; Levacetylmethadol; Levargorphan; Levoisomethadone; Levomethadone; Levomethorphan; Levomoramide; Levophenacylmorphan; Levopropoxyphene; Levorphanol; Lofentanil; Loperamide; LPK-26; LS-115509; Lufuradom; Matrine; MCOPPB; Menthol; Meperidine-N-oxide; Meptazinol; Metazocine; Metethoheptazine; Methadone; Metheptazine; Methorphan (racemethorphan); Methyldesorphine; Methyldihydromorphine (dihydroheterocodeine); Methyldihydromorphinone; Methylketobemidone; Metofoline; Metonitazene; Metopon; Mirfentanil; Mitragynine; Mitragynine pseudoindoxyl; Morphanol (racemorphanol); Morphenol; Morpheridine; Morphine; Morphine methylbromide; Morphine-6-glucuronide; Morphine-N-oxide; Morphine-N-oxide (genomorphine); Morphinone; Morphol; Moxazocine; MT-45; MT-7716; Myrophine; Nalbuphine; Nalbuphone; Nalfurafine; Nalorphine; Nalorphine dinicotinate; Naltrexol; N-cyclopropylmethylnoretorphine; Nepenthone; Nexeridine; Nicocodeine; Nicodicodeine; Nicomorphine; N-Methylcarfentanil; N-Methylmorphinan; NNC 63-0532; Noracymethadol; Norbuprenorphine; Norbuprenorphine-3-glucuronide; Norcodeine; Noribogaine; Norlevorphanol; Normethadone; Normorphine; Noroxymorphone; Norpipanone; Norpropoxyphene; Nortilidine; N-Phenethyl-14-ethoxymetopon; N-Phenethyl-14-ethoxymetopon; N-Phenethylnordesomorphine; N-Phenethylnormorphine; Ocfentanil; O-Desmethyltramadol; Ohmefentanyl; Opium; Oripavine; Oxilorphan; Oxpheneridine (carbamethidine); Oxycodone; Oxymorphazone; Oxymorphol; Oxymorphone; Pantopon; Papaveretum (Omnopon); Parafluorofentanyl; Pentamorphone; Pentazocine; PEPAP; Pericine; Pethidine (meperidine); Phenadone; Phenadoxone (heptazone); Phenampromide; Phenaridine; Phenazocine; Phencyclidine; Pheneridine; Phenomorphan; Phenoperidine; Pholcodine (morpholinylethylmorphine); Picenadol; Piminodine; Piperidylthiambutene; Piritramide; Prodilidine; Profadol; Proglumide; Proheptazine; Properidine (ipropethidine); Propiram; Propylketobemidone; Prosidol; Proxorphan; Pseudoakuammigine; Pseudomorphine; Pyrrolidinylthiambutene; Pyrroliphene; PZM21; Quadazocine; R-30490; R-4066; Racemoramide; RAM-378; Remifentanil; Ro-1539; Ro4-1539; Ro64-6198; Ro65-6570; RWJ-394,674; Salvinorin A; Salvinorin B ethoxymethyl ether; Salvinorin B methoxymethyl ether; Sameridine; SB-612,111; SC-17599; Semorphone; SKF-10047; SNC-80; SoRI-9409; Spiradoline; SR-16435; SR-8993; Sufentanil; TAN-67; Tannagine; Tapentadol; Tetrapon; Thebacon; Thebacon (acetyldihydrocodeinone, dihydrocodeinone enol acetate); Thebaine; Thenylfentanyl; Thevinone; Thiambutene; Thiazocine; Thienorphine; Thiobromadol (C-8813); Thiofentanyl; Tifluadom; Tilidine; Tonazocine; Tramadol; Transisocodeine; Trefentanil; Trimebutine; Trimeperidine (promedol); U-47700; U-50,488; U-69,593; Viminol; Volazocine; Zenazocine; α-Chlorocodide; α-Chloromorphide; α-hydrocodol; α-Methylacetylfentanyl; α-Methylfentanyl; α-Methylthiofentanyl; β-Chlorocodide; β-hydroxyfentanyl; β-hydroxythiofentanyl; β-Methylfentanyl; ψ-Akuammigine; derivatives thereof; and combinations thereof.

In some examples, the device is implantable in a subject. In some examples, the device is anatomically designed for the subject.

In some examples, the bioresorbable polymer is configured to be stable for an amount of time after the device is implanted in the subject. As used herein, “stable” means that 10 wt % or less (e.g., 9% or less, 8% or less, 7% or less, 6% or less, 5% or less, 4% or less, 3% or less, 2% or less, or 1% or less) of the bioresorbable polymer is resorbed over the selected time period after the device is implanted in the subject.

In some examples, the bioresorbable polymer is configured to be stable for an amount of time of 2 weeks or more after the device is implanted in the subject (e.g., 3 weeks or more, 4 weeks or more, 5 weeks or more, 6 weeks or more, 7 weeks or more, 8 weeks or more, 9 weeks or more, 10 weeks or more, or 11 weeks or more). In some examples, the bioresorbable polymer is configured to be stable for an amount of time of 12 weeks or less after the device is implanted in the subject (e.g., 11 weeks or less, 10 weeks or less, 9 weeks or less, 8 weeks or less, 7 weeks or less, 6 weeks or less, 5 weeks or less, 4 weeks or less, or 3 weeks or less). The amount of time for which the bioresorbable polymer is configured to be stable can range from any of the minimum values described above to any of the maximum values described above. For example, the bioresorbable polymer can be configured to be stable for an amount of time of from 2 weeks to 12 weeks after the device is implanted in the subject (e.g., from 2 weeks to 7 weeks, from 7 weeks to 12 weeks, from 2 weeks to 4 weeks, from 4 weeks to 6 weeks, from 6 weeks to 8 weeks, from 8 weeks to 10 weeks, from 10 weeks to 12 weeks, from 3 weeks to 12 weeks, from 2 weeks to 11 weeks, from 3 weeks to 11 weeks, from 6 weeks to 12 weeks, from 6 weeks to 8 weeks, from 9 weeks to 12 weeks, from 7 weeks to 12 weeks, from 6 weeks to 11 weeks, from 7 weeks to 11 weeks, or from 7 weeks to 9 weeks).

In some examples, the bioresorbable polymer can be quicky absorbed and/or can have a porosity allowing for vascularization to begin within the first 24 hours. For example, some cells have a high metabolic requirement, and the device can comprise a bioresorbable polymer that can be quicky absorbed and/or can have a porosity allowing for vascularization to begin within the first 24 hours to support the high metabolic requirement of such cells (e.g., muscle cells, stem cells, mixtures, etc.).

In some examples, the bioresorbable polymer is configured to be stable for an amount of time of 1 hour or more after the device is implanted in the subject (e.g., 2 hours or more, 3 hours or more, 4 hours or more, 5 hours or more, 6 hours or more, 8 hours or more, 10 hours or more, 12 hours or more, 16 hours or more, 20 hours or more, 24 hours or more, 30 hours or more, 36 hours or more, 42 hours or more, 48 hours or more, 54 hours or more, 60 hours or more, 66 hours or more, 3 days or more, 3.5 days or more, 4 days or more, 4.5 days or more, 5 days or more, 5.5 days or more, 6 days or more, 6.5 days or more, 7 days or more, 8 days or more, 9 days or more, 10 days or more, 11 days or more, 12 days or more, or 13 days or more). In some examples, the bioresorbable polymer is configured to be stable for an amount of time of 2 weeks or less after the device is implanted in the subject (e.g., 13 days or less, 12 days or less, 11 days or less, 10 days or less, 9 days or less, 8 days or less, 7 days or less, 6.5 days or less, 6 days or less, 5.5 days or less, 5 days or less, 4.5 days or less, 4 days or less, 3.5 days or less, 3 days or less, 66 hours or less, 60 hours or less, 54 hours or less, 48 hours or less, 42 hours or less, 36 hours or less, 30 hours or less, 24 hours or less, 20 hours or less, 16 hours or less, 12 hours or less, 10 hours or less, 8 hours or less, or 6 hours or less). The amount of time for which the bioresorbable polymer is configured to be stable can range from any of the minimum values described above to any of the maximum values described above. For example, the bioresorbable polymer can be configured to be stable for an amount of time of from 1 hour to 2 weeks after the device is implanted in the subject (e.g., from 1 hour to 24 hours, from 24 hours to 2 weeks, from 1 hour to 6 hours, from 6 hours to 12 hours, from 12 hours to 24 hours, from 24 hours to 1 week, from 1 week to 2 weeks, from 6 hours to 2 weeks, from 24 hours to 2 weeks, from 1 hour to 1 week, from 1 hour to 24 hours, or from 6 hours to 1 week).

In some examples, the second bioresorbable polymer is configured to be stable for a second amount of time after the device is implanted in the subject. As used herein, “stable” means that 10 wt % or less (e.g., 9% or less, 8% or less, 7% or less, 6% or less, 5% or less, 4% or less, 3% or less, 2% or less, or 1% or less) of the second bioresorbable polymer is resorbed over the selected time period after the device is implanted in the subject.

In some examples, the second bioresorbable polymer is configured to be stable for an amount of time of 2 weeks or more after the device is implanted in the subject (e.g., 3 weeks or more, 4 weeks or more, 5 weeks or more, 6 weeks or more, 7 weeks or more, 8 weeks or more, 9 weeks or more, 10 weeks or more, or 11 weeks or more). In some examples, the second bioresorbable polymer is configured to be stable for an amount of time of 12 weeks or less after the device is implanted in the subject (e.g., 11 weeks or less, 10 weeks or less, 9 weeks or less, 8 weeks or less, 7 weeks or less, 6 weeks or less, 5 weeks or less, 4 weeks or less, or 3 weeks or less). The amount of time for which the second bioresorbable polymer is configured to be stable can range from any of the minimum values described above to any of the maximum values described above. For example, the second bioresorbable polymer can be configured to be stable for an amount of time of from 2 weeks to 12 weeks after the device is implanted in the subject (e.g., from 2 weeks to 7 weeks, from 7 weeks to 12 weeks, from 2 weeks to 4 weeks, from 4 weeks to 6 weeks, from 6 weeks to 8 weeks, from 8 weeks to 10 weeks, from 10 weeks to 12 weeks, from 3 weeks to 12 weeks, from 2 weeks to 11 weeks, from 3 weeks to 11 weeks, from 6 weeks to 12 weeks, from 6 weeks to 8 weeks, from 9 weeks to 12 weeks, from 7 weeks to 12 weeks, from 6 weeks to 11 weeks, from 7 weeks to 11 weeks, or from 7 weeks to 9 weeks).

For example, the bioresorbable polymer and the second bioresorbable polymer can be configured to be stable for the same amount of time or for different amount of time. In some examples, the bioresorbable polymer can be configured to be stable for a longer amount of time than the second bioresorbable polymer, or vice-versa.

In some examples, the device can further comprise a bioactive coating, for example on a surface of the device, for example to promote cell adhesion, cell proliferation, tissue integration, or a combination thereof.

In some examples, the device is produced by additive manufacturing (e.g., 3D printing). In some examples, the device can be produced by subtractive manufacturing.

As printing technology evolves, the resolution of printing can produce smaller channels which could serve as vascularized chambers for cells that have a higher metabolic requirement than adipose cells, thereby having closer proximity to the vascular encapsulated wall.

In some examples, the device can be produced using a mold.

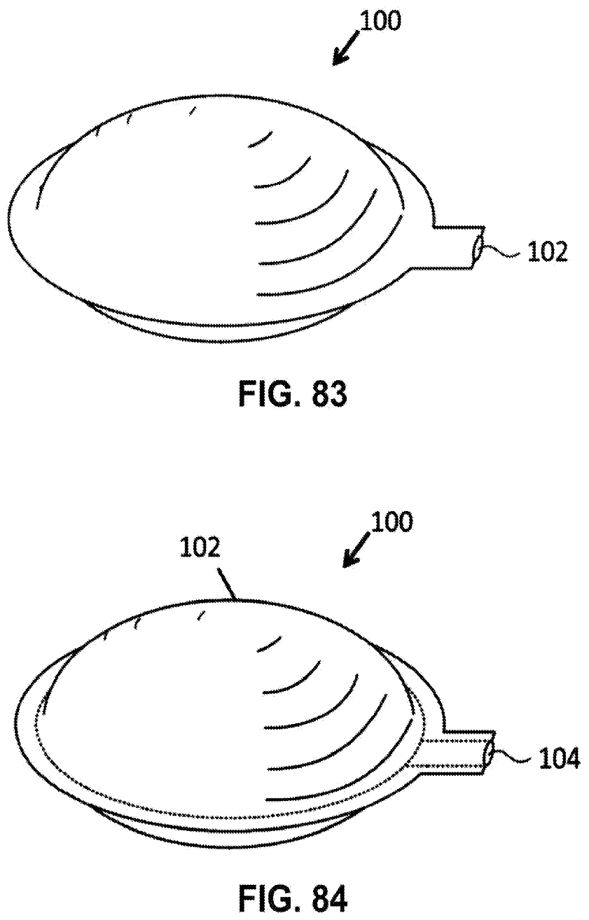

Referring now to FIG. 83, disclosed herein are devices 100 configured to be inserted into an anatomical location of a subject, the device comprising a bioresorbable polymer 102. When the device is inserted into the anatomical location of the subject, then device becomes encapsulated by vascular tissue and the bioresorbable polymer is resorbed over an amount of time, thereby forming a chamber in place of the bioresorbable polymer; wherein the chamber is defined by a boundary formed from the vascular tissue; wherein the chamber is perfusable; and wherein the chamber is configured to be at least partially filled with a first plurality of cells.

Referring now to FIG. 84, in some examples, the device, before the bioresorbable polymer is resorbed, further includes a compartment 104 defined by the bioresorbable polymer, wherein the compartment is configured to be at least partially filled with a second plurality of cells before or shortly after being inserted into the anatomical location of the subject. In some examples, the compartment is non-filled (e.g., empty) during insertion and is subsequently at least partially filled after insertion.

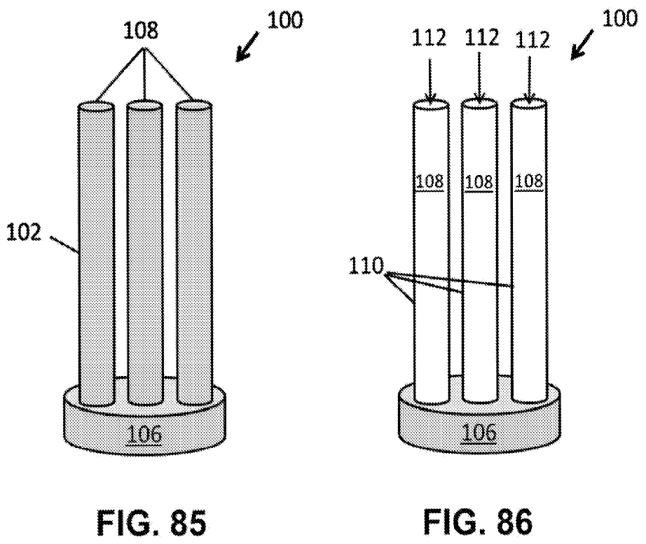

Referring now to FIG. 85, in some examples, the device 100 comprises a plug 106 comprising a bioresorbable polymer; and a plurality of conduits 108 comprising the bioresorbable polymer. When the device is inserted into the anatomical location of the subject, then device becomes encapsulated by vascular tissue and the bioresorbable polymer is resorbed over an amount of time, thereby forming a plurality of channels in place of the plurality of conduits 108 and a port in place of the plug 106; wherein each channel is defined by a boundary formed from the vascular tissue; wherein the port is further defined by the vascular tissue; wherein the port and the plurality of channels are perfusable; wherein each of the plurality of channels is fluidly connected to the port; and wherein the port is configured to receive cells, such as adipose tissue, and the plurality of channels are configured to be at least partially filled with cells, such as adipose tissue, delivered through the port.

Referring now to FIG. 86, in some examples, the plurality of conduits 108 are hollow, such that each of the plurality of conduits 108 comprises a wall 110 defining a lumen 112, the wall 110 comprising the bioresorbable polymer, and each channel is defined by an inner wall and an outer wall, the inner wall and the outer wall being vascular tissue.

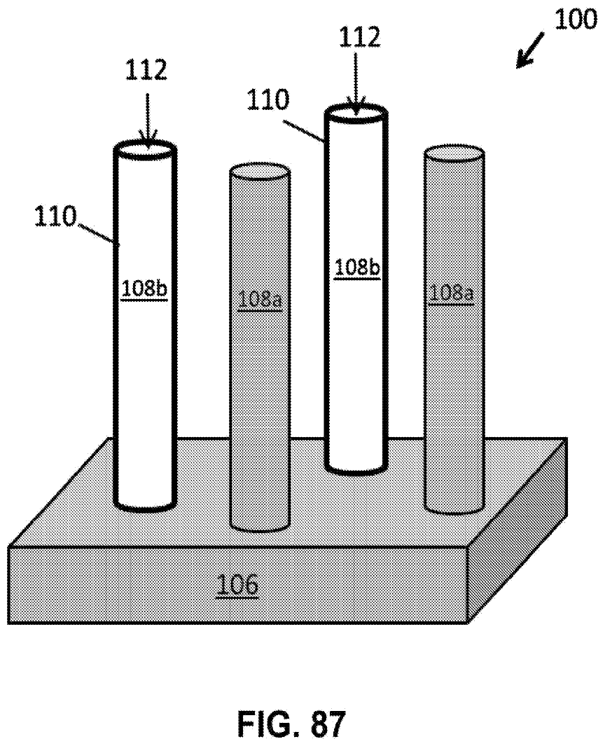

Referring now to FIG. 87, in some examples, the plurality of conduits 108 comprises: a first population of conduits 108a; and a second population of conduits 108b; each of the first population of conduits and the second population of conduits including one or more of the plurality of conduits; such that the plurality of channels comprises: a first population of channels; and a second population of channels; each of the first population of channels and the second population of channels including one or more of the plurality of channels; wherein the first population of conduits 108a are solid, such that boundary defining each of the first population of channels is an outer wall; and wherein the second population of conduits 108b are hollow such that the each of the second population of conduits 108b comprises a wall 110 defining a lumen 112, the wall 110 comprising the bioresorbable polymer, and each of the second population of channels is defined by an inner wall and an outer wall, the inner wall and the outer wall being vascular tissue.

### Methods

Also disclosed herein are methods of manufacturing any of the devices disclosed herein. For example, the methods can comprise making the device using additive manufacturing (e.g., 3D printing). In some examples, the device can be produced by subtractive manufacturing.

In some examples, the method comprises making the device based on a 3D model. In some examples, the 3D model is based on the Fibonacci equation.

In some examples, the 3D model is based on an anatomical image of a subject. In some examples, the method further comprises collecting the anatomical image of the subject.

As printing technology evolves, the resolution of printing can produce smaller channels which could serve as vascularized chambers for cells that have a higher metabolic requirement than adipose cells, thereby having closer proximity to the vascular encapsulated wall.

Also disclosed herein are methods of making any of the devices disclosed herein, the method comprising forming a mold with desired dimensions and features, injecting a bioresorbable polymer into the mold to create a solid structure optionally including one or more compartments, optionally incorporating conduits and/or ports within the mold, and removing the mold to obtain the device comprising the bioresorbable polymer including one or more perusable compartments, conduits, and/or ports.

Also disclosed herein are methods of treating a subject in need thereof, the methods comprising implanting the device into the subject.

In some examples, the device is at least partially filled with a nutrient medium, for example to support cell growth and functionality.

In some examples, the method comprise implanting an empty device and subsequently at least partially filling the device with a nutrient medium, cells, adipose tissue, a mixture, etc. after implantation (before and/or after the bioresorbable polymer has resorbed). In some examples, the device can be filled at multiple timepoints after implantation.

In some examples, the device is at least partially filled with cells before or after implantation. The cells can be any type of cells, such as mixtures of different types of cells. The cells can, for example, comprise adipose tissue, muscle cells, stem cells or progenitor cells capable of differentiating into adipose tissue or other desired cell types, or a combination thereof.

In some examples, the device is at least partially filled with adipose tissue before or after implantation, such as autologous adipose tissue. In some examples, the device is at least partially filled with a mixture comprising adipose tissue before or after implantation. The mixture can, for example, further comprise an additional component, which can, for example, improve the uptake of the cells (e.g., fat) by the subject. For example, the additional component can comprise platelets, plasma, platelet-rich plasma (PRP), stem cells, a protein, or a combination thereof.

In some examples, the device is implanted into at least a portion of a breast of the subject, for example mammary gland, subcutaneous tissue, pectoral muscle, or a combination thereof. In some examples, the device is implanted into a breast of the subject. For example, the method can comprise full or partial breast reconstruction or augmentation, for example after a lumpectomy or mastectomy, restoring breast volume after a lumpectomy or mastectomy, correcting breast asymmetry, enhancing breast shape or contour, providing support or reinforcement to the breast tissue, improving breast aesthetics, or a combination thereof.

In some examples, the method further comprises anatomically designing the device for the subject.

In some examples, the methods can further comprise treatment of an oncological disorder, such as breast cancer. In some examples, the devices can further include a therapeutic agent, for example for treatment of the oncological disorder.

For the treatment of oncological disorders, the devices disclosed herein can be administered to a patient in need of treatment in combination with other antitumor or anti-cancer substances and/or with radiation and/or photodynamic therapy and/or with surgical treatment to remove a tumor. These other substances or treatments can be given at the same as or at different times from the devices disclosed herein. For example, the devices disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Biobreast: 3D Bioengineered Breast Restoration

Disclosed herein are devices that utilize the body's natural tendency to make a scar around (e.g., encapsulate) an implanted material, which it does to separate itself from the foreign substance. This scarring/encapsulation process has traditionally been regarded as problematic in the case of implants, since the permanent prosthetic implant material causes inflammation and the capsule thickens, resulting in contracture of the scar. The implant becomes firm and may be painful due to the tight scar around it. However, one feature of the resulting scar is the vascularized layer of tissue that is formed. As described herein, this vascularized scar tissue can be the source of a capillary network that can then support fat transfer.

An important consideration in transferring adipose tissue is the need for a blood supply within a certain proximity in order to nourish the graft so that it remains viable. Standard fat grafting approaches rely on even distribution of fat into the tissue to maintain fat take. In the approach described herein, the body's natural propensity to create a layer of vascularized scar tissue can be harnessed to support the creation of a three dimensional matrix for structured adipose cell transfer. This process allows formation of 3 dimensional tissue for replacement or augmentation of breast tissue (or tissue elsewhere in the body) without any long term use of foreign material. For example, the process described herein can include the use of devices, e.g., an engineered three dimensional construct, made from biodegradable/bioresorbable polymers, which offers the possibility of tissue creation without any permanent foreign material. Collagen can be added (which is a permanent material) if desired for additional structural support but is not required.

In some examples, the technology described herein relates to restoration of the breast parenchyma by 3D printing a construct with resorbable polymer materials (e.g., 3D BioBreast) for example into a three-dimensional parametric teardrop shape, e.g. following the Fibonacci equation. The construct comprises channels (e.g., circular channels) printed using polymers with biodegradable properties that create incapsulated vascular channels for the eventual delivery of the adipose cells, thereby facilitating in-growth.

The vascular channels can be formed using different options. In a first option, the channels can be hollow, in which case the fat is injected at the time of surgery, and then, as the channel wall gets absorbed, there is capillary ingrowth and capsular formation. In a second option, the channel can be solid, in which case, the fat is injected in a second surgery stage after the solid material gets resorbed, an encapsulated channel would form. The hollow channel option can facilitate capsulate growth more readily. However, the solid channel option may be preferable in certain situations, for example depending on a property of the biodegradable material being used.

The ability to design, print, and create a biological construct would be a revolutionary advancement for breast reconstruction as well as breast enhancement. Currently, implants made out of silicone are traditionally used, but recently received a black box warning by the FDA, due to risk of implant failure, a rare form of ALCL that is associated with some textured implants, and Breast implant illness. The silicone implant technology has not advanced substantially over the past three decades. The current alternative is autologous reconstruction, which permits restoration of the breast with adipose tissue from a donor site, by microsurgically connecting the blood supply. However, this is a more complex operation, requires longer recovery and not every patient has excess tissue available for this purpose.

Two example materials that can be considered for the formation of the BioBreast vascular channels are polymers and/or collagen.

The process of making a 3D BioBreast can comprise printing with a selected polymer, collagen, or bio-ink into a three-dimensional shape, such as a parametric teardrop shape. The 3D printed construct is then inserted into the breast pocket to form an encapsulated channel system that uses the body's natural capsule making capabilities to develop similar capsule-like channels which have vascular ingrowth. Using a polymer/collagen/bio-ink that resorbs as the capsule is formed can ultimately result in a channel to house fat and facilitate its fat grafting viability. This would allow for the body's natural capsule forming capability, which in itself is vascularized, to create a network of channels for the fat. This can be done with a hallow channel with the fat within the channel or at a second stage once the channel is fully formed, the channel would be solid in those cases. The printed construct would be reabsorbed—it would depend on the chosen materials and the reabsorption rate to see which would be most optimal.

By injecting into the uniform encapsulated channels, the surgeon would have control of the amount of fat and stroma being injected into the breast area. The combination of fat and stromal matrix injection process would depend on the volume of the desired breast size. However, in the case of a mastectomy, it may take a second fat grafting session to ensure the breast volume is accomplished. The purpose for using high-quality fat grafting matrix is due to the collagen type I-IV and other Extracellular matrices (ECM) components it contains, making it an ideal source as a cell adhesion receptor, particularly in tissue engineering.

The shape of the 3D printed construct can, for example, be designed using a parametric equation applied to a computational design. For example, the design can allow for customization of different variables of the breast shape depending on the characteristics of the material that are being used. In some examples, the design can be fully adjustable to the patient's anatomy. The design can then be sent to a 3D printer for printing.

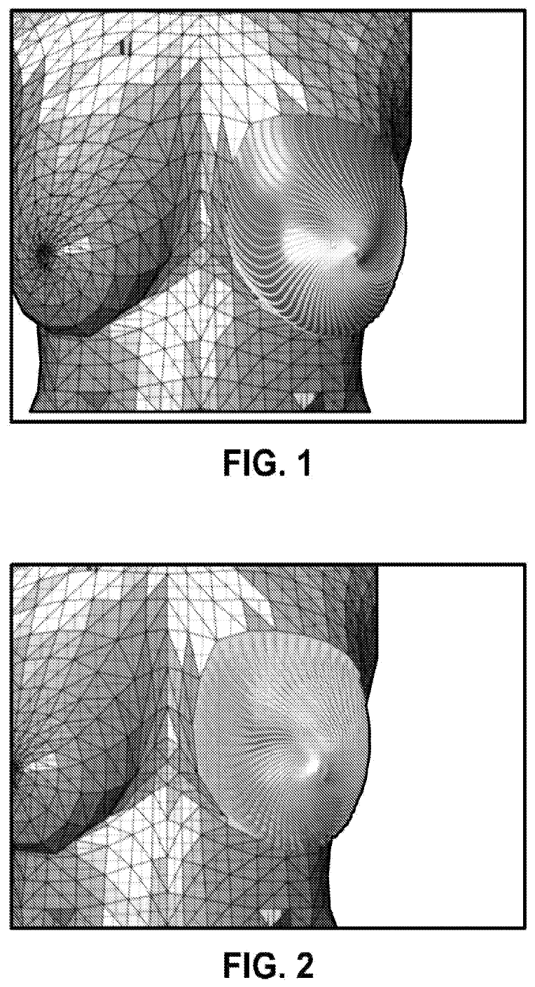

Schematic diagrams illustrating an example BioBreast design based on a free form curve model are shown in FIG. 1-FIG. 2.

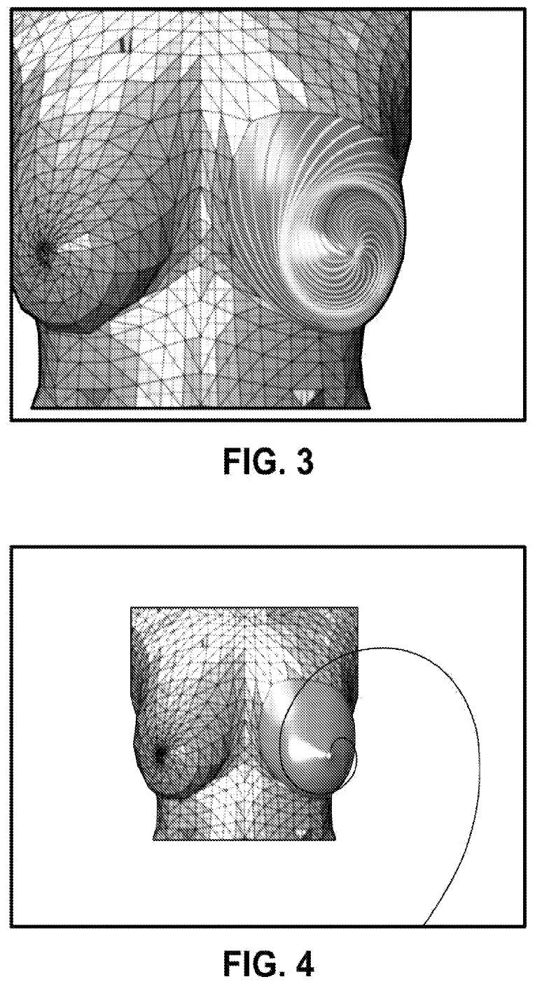

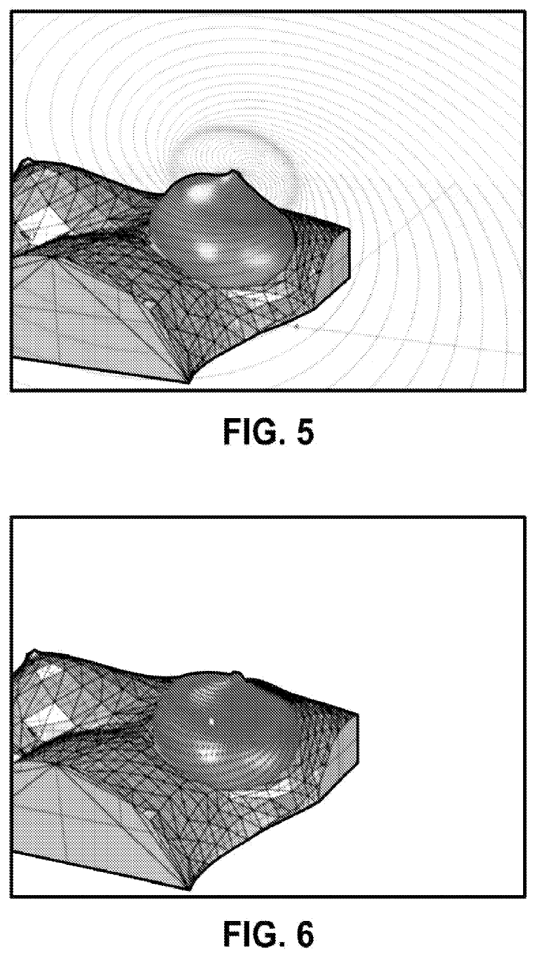

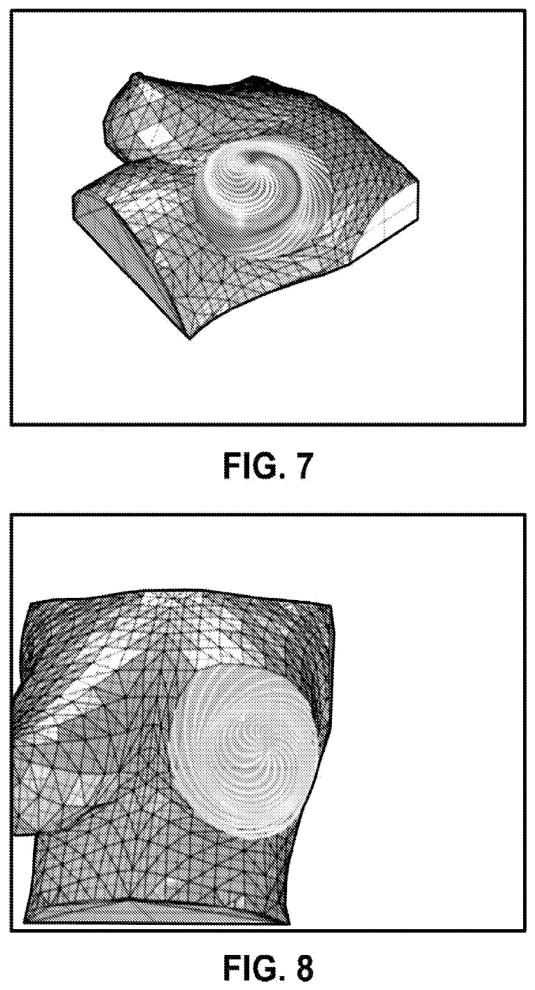

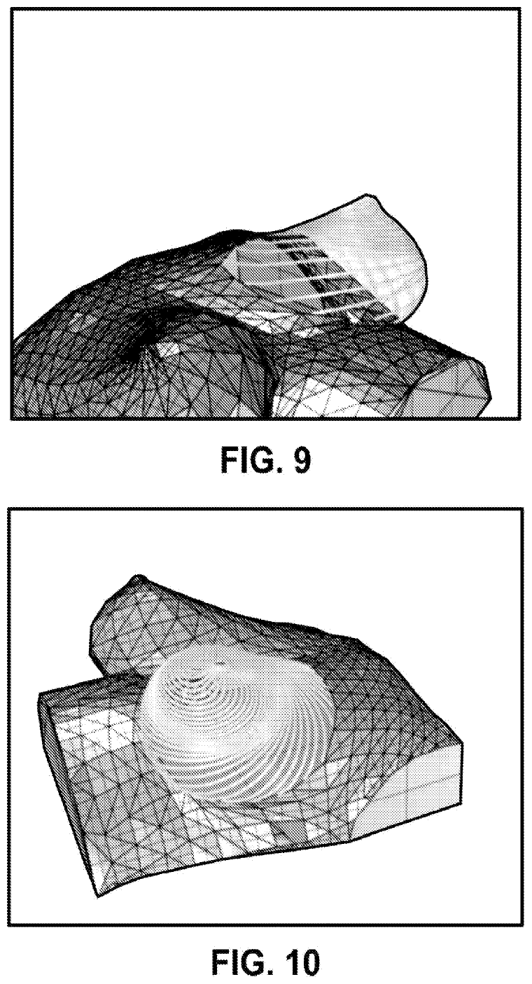

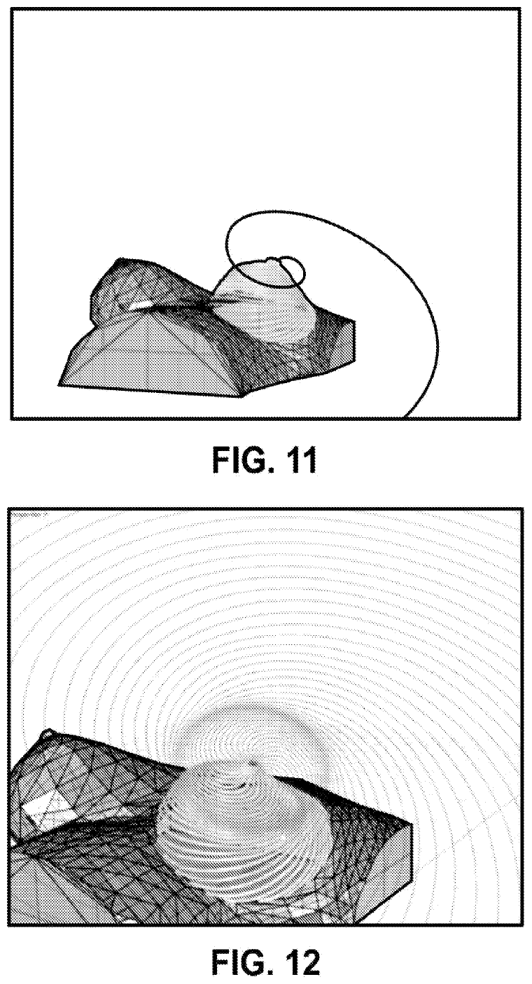

Schematic diagrams illustrating an example BioBreast design based on a Fibonacci model are shown in FIG. 3-FIG. 12.

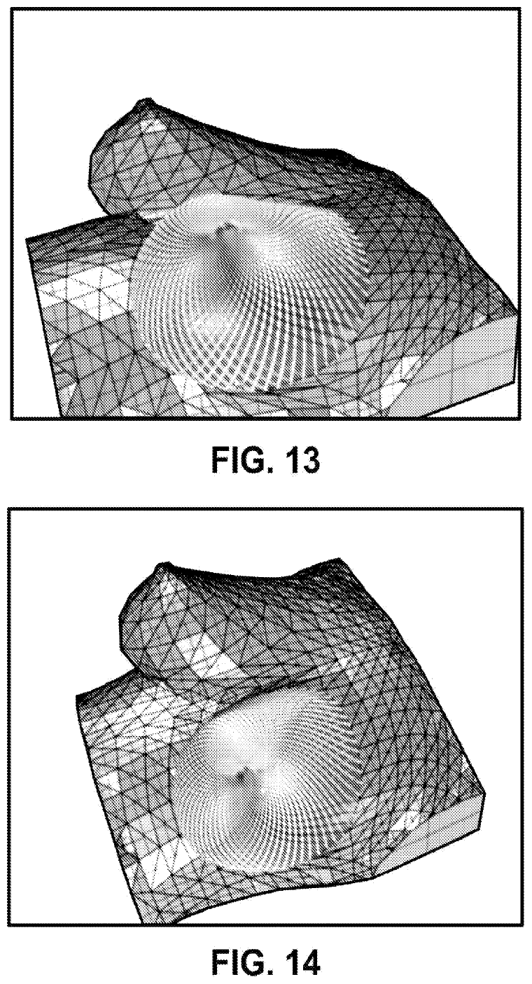

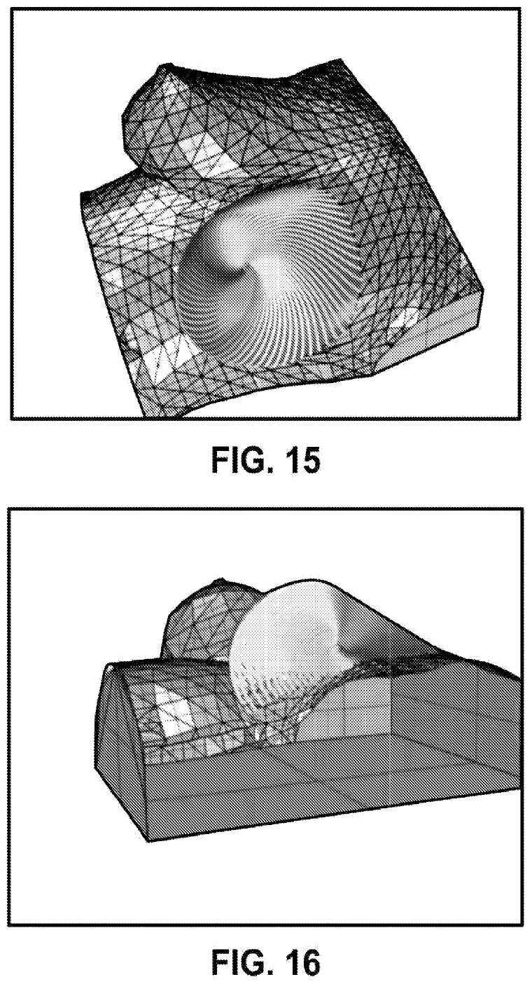

Schematic diagrams illustrating an example BioBreast modeled using a combination approach are shown in FIG. 13-FIG. 15.

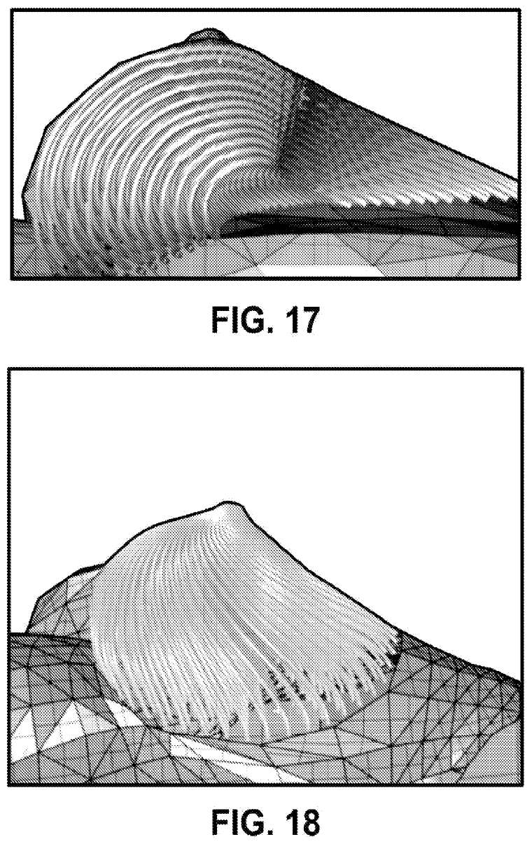

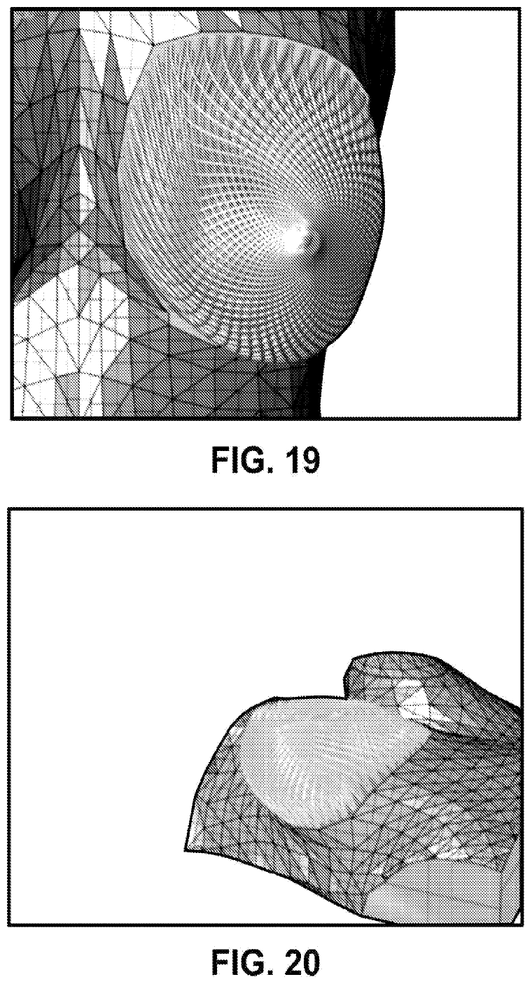

Schematic diagrams illustrating an example of the internal matrix of a BioBreast are shown in FIG. 16-FIG. 20.

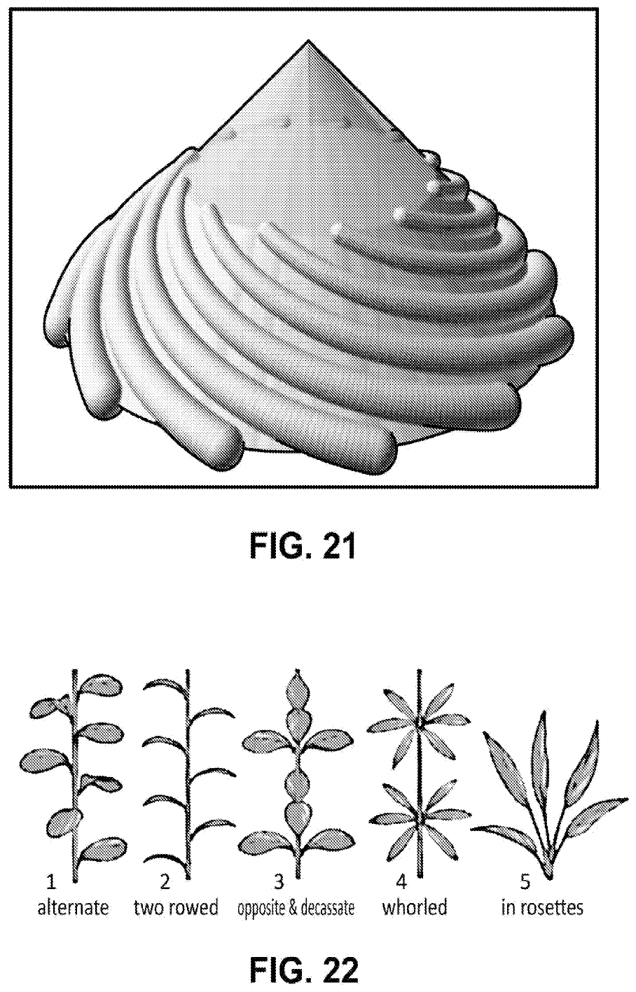

A schematic diagram illustrating an example design for a BioBreast using linearly oriented channels with a larger diameter is shown in FIG. 21. In some examples, the BioBreast can further comprise concentric sets of channels such as those shown in FIG. 21, wherein the diameter of each set of channel can vary from one set to the next, e.g., the diameter can increase or decrease from the outermost concentric set to the innermost concentric set, or vice versa.

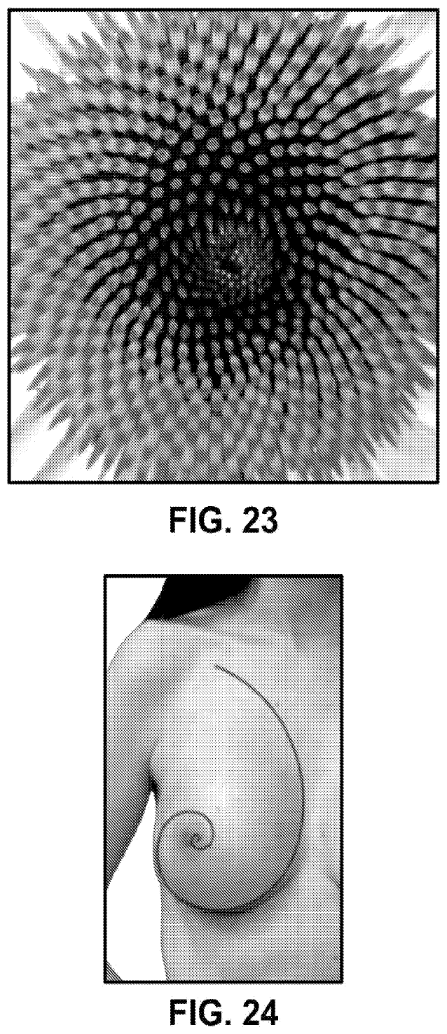

Another design option can be based on phyllotaxis. Phyllotaxis/phyllotaxy is the arrangement of leaves on a plant stem & the phyllotactic spirals form a distinctive class of patterns in nature, examples are shown in FIG. 22 and FIG. 23 (see also https://goldenratiomyth.weebly.com/phyllotaxis-the-fibonacci-sequence-in-nature.html). For example, additional lobule-like pockets would be printed along the channel to allow for a larger volume of fat. The fat and stromal matrix would be injected as applied to the phyllotaxis design into the bio absorbable uniform vascular channels.

Primary and secondary channels could be designated within the inner and outer area of the BioBreast.

A Fibonacci sequence shown in FIG. 23 is an example of how the vascular channels can be arranged.

FIG. 24 represents the Fibonacci sequence used to create a teardrop shape in the anterior-posterior and used as a guide. The parametric equation was used in an adjustable CAD 3D model using Grasshopper software. So it has customized ability of the different shape variables depending on material and qualities.

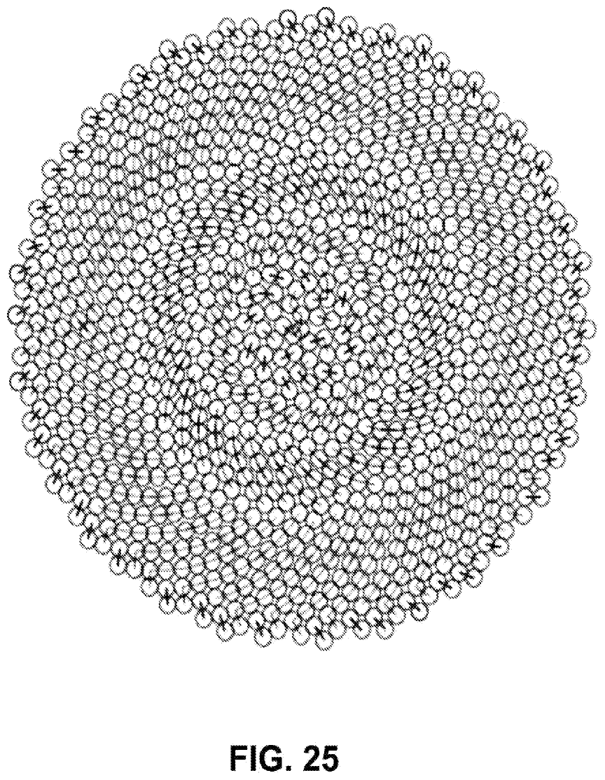

FIG. 25 is Fibonacci sequence. This circular model was redesigned using the computational software into an oval shape and later used to create the natural teardrop shape in the anterior posterior of the breast.

The 3D BioBreast would revolutionize breast reconstruction and potentially also breast enhancement procedures by eliminating the current silicone implants. Implants problems include—infection, potentially leading to rare cancers (ALCL—anaplastic large-cell lymphoma) and breast implants illness. Ultimately, this combines the power of harvesting adipose tissue, which is usually ubiquitous to construct a bioengineered breast gland without using more complicated microvascular procedures that use donor tissue from other areas of the body and require a longer healing time for the patient.

The 3D BioBreast would provide a new paradigm approach to breast reconstruction. The BioBreast would feel more organic and allow the patient to harness their healing ability and promote fat-growth regeneration. The technology would also enable breast customization with more of a natural shape to the original breast.

The technology can also be extended to other parts of the body, for example where a scan can be performed and a tissue volume be enhanced and/or a tissue volume defect be corrected (e.g., buttocks) with the engineered three dimensional constructs described herein.

The technology described herein can also be used for developing and producing skin for local treatment and wound healing.

### Example 2

The technology disclosed herein envisions a 3D microvascular printed network supporting blood flow to a fat cell.

Relative to other methods, it would be simpler to have a construct with vascular in-growth. The idea herein utilizes the body's natural capability of making vascularized capsules around a biodegradable implant material to form channels for fat grafting and in-growth. A 3D printed biodegradable polymer would facilitate the creation of vascularized capsule channels in which the adipose cells could be injected. This way, the surgeon is utilizing the body as a natural capsule formation to augment body tissue.

The idea is to print a 3D BioBreast comprising polymer bio-ink and/or collagen material into a three-dimensional shape, such as a parametric teardrop shape, for example following the Fibonacci equation.

An example of this concept would be like a leaf on the tree, sort of printing tree branches. Further, in some examples, instead of just printing the “branch” the devices and methods herein can further include printing “leaves” on the branch, which would increase the fat volume, while maintaining proximity to blood supply.

The 3D printed construct would then be inserted into the breast pocket to form an encapsulated channel system by using the body's natural capsule making capabilities to develop similar capsule-like channels which have vascular ingrowth. Fat would be injected into the vascular vessel system creating a self-generating natural bio-implant.

The 3D BioBreast can promote fat-in growth regeneration and quicker healing time.

### Example 3

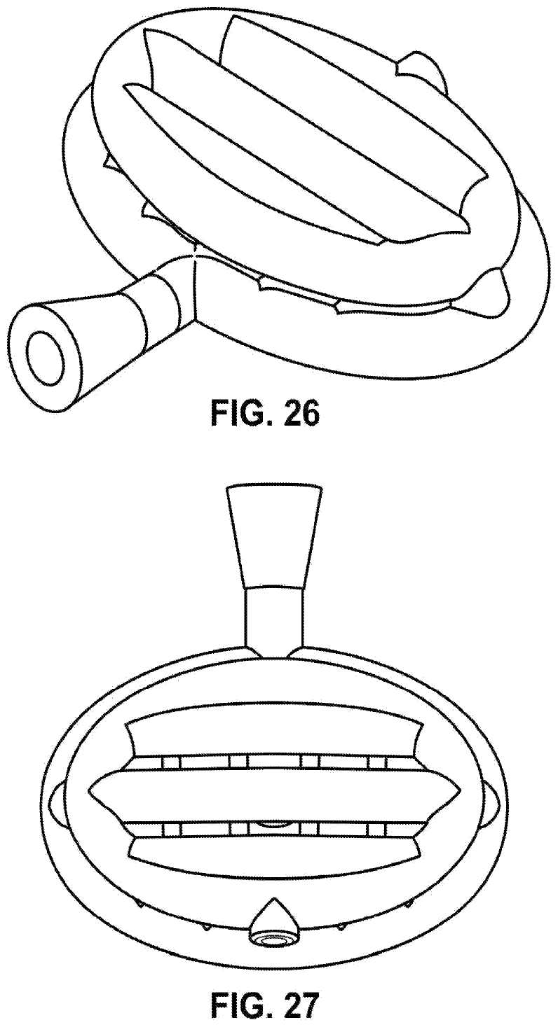

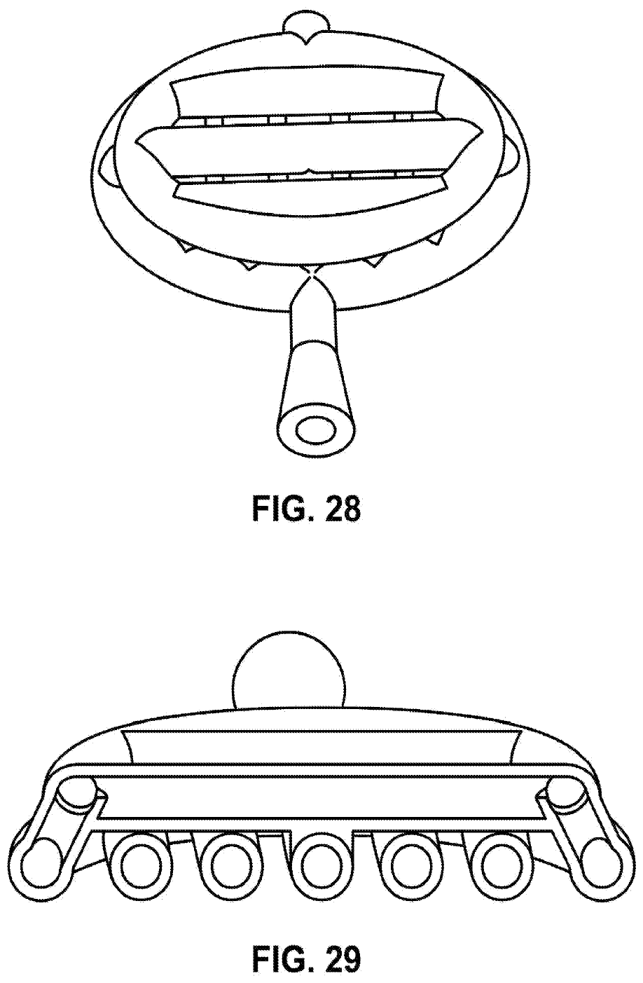

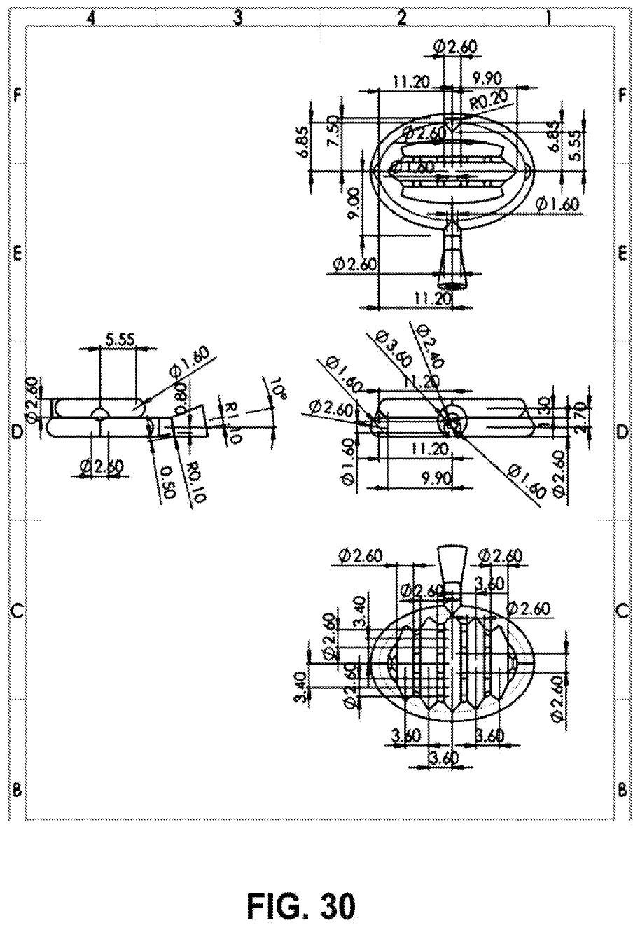

An example BioBreast device was designed using CAD, as shown schematically in FIG. 26-FIG. 30. This example device was dimensioned for implantation into a rat.

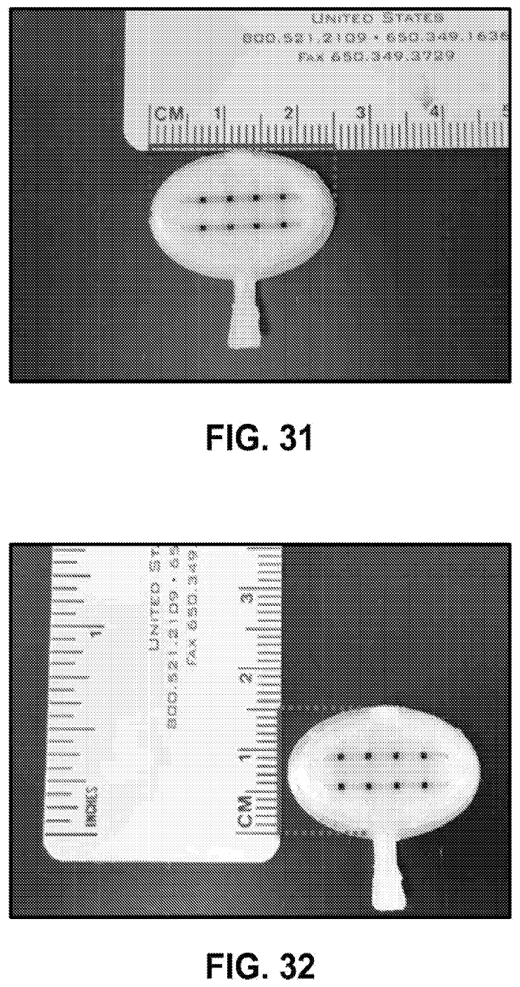

An example device of this design was fabricated using 3D printing. The printing parameters of the example device are summarized in Table 1. Photographs of the example device are shown in FIG. 31 and FIG. 32.

TABLE 1
Printing parameters.

| Infill Density | 20% | |
|---|---|---|
| Printing Temperature | 210° | C. |
| Print Velocity | 40 | mm/s |
| Diameter of Printhead used | 0.40 | mm |
| Printing Time | 27 | min |

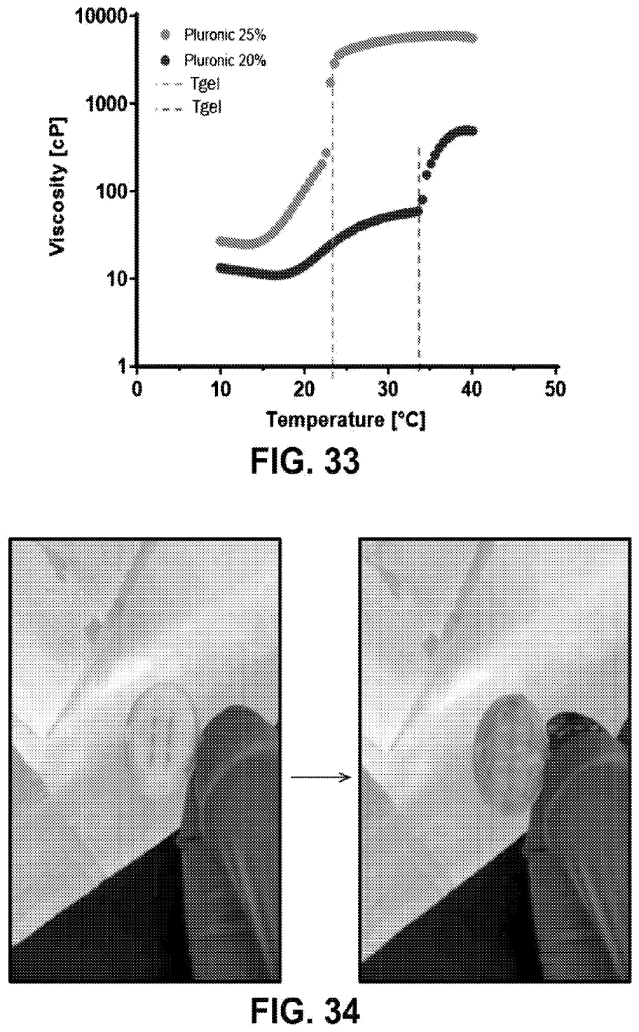

The viscosity of different polymers for the device were assessed. A thermos-responsive hydrogel was used for injection feasibility. Thus, different viscosities are obtainable by increasing the temperature, as shown in FIG. 33. Based on these results, Pluronic at 25% w/v concentration was selected, as it becomes a stiff gel at room temperature, making injection feasible (FIG. 34).

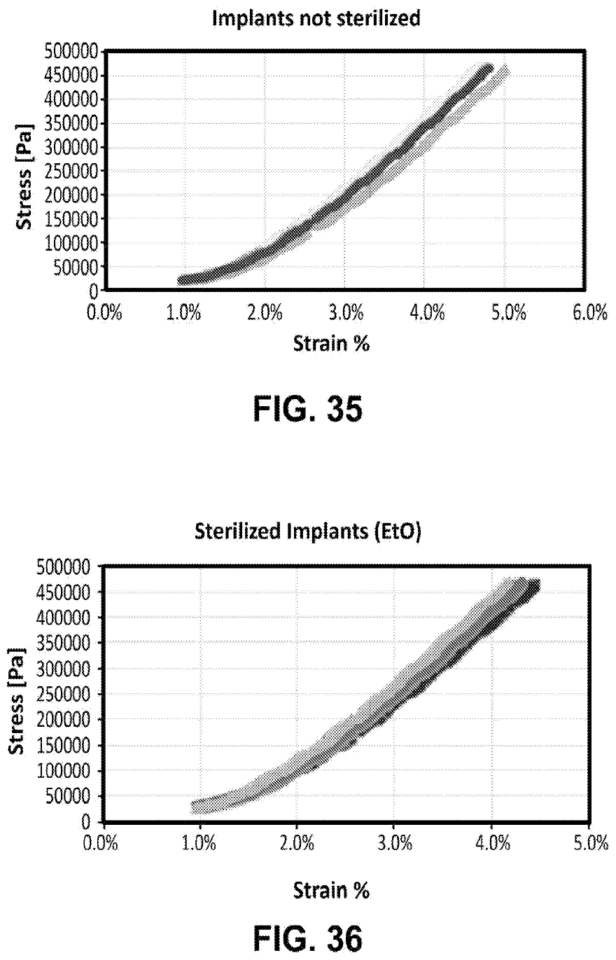

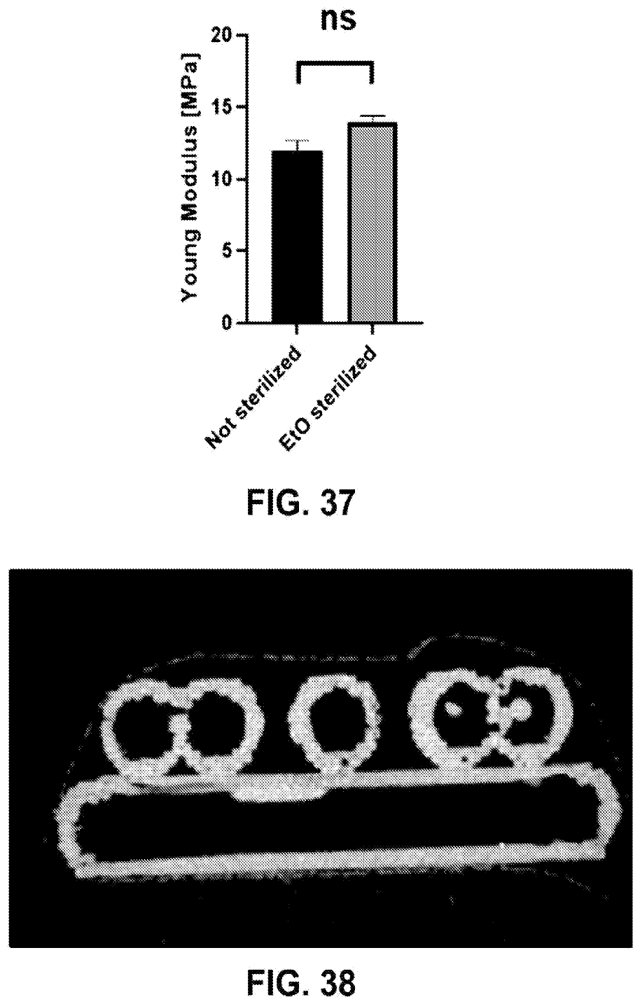

The mechanical properties were characterized for sterilized and non-sterilized devices (FIG. 35-FIG. 37). The results indicate that sterilization with EtO did not significantly affect the mechanical properties of the devices (FIG. 35-FIG. 37).

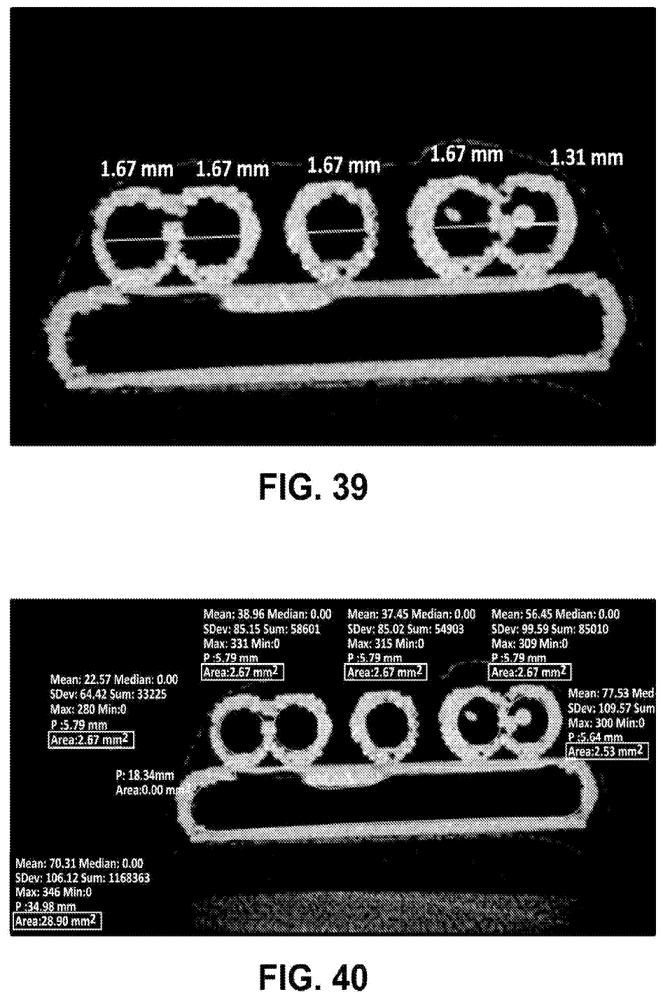

In some examples, the device can be fabricated using a polymer that is radio-opaque, such that microCT can be used for assessment of the internal structure of the device. An example microCT image of an example device is shown in FIG. 38, which shows a hollow shape with two layers and an theoretical internal diameter of 1.6 mm. An image showing measurements of the internal diameters of the tubes is shown in FIG. 39. There are possible difference in measuring diameter between operators. An image showing measurements of the empty surface of the hollow scaffolds is shown in FIG. 40. These measurements can be used to derived the volume to fill the device. The filling volume was estimated to be 0.44 mL for each scaffold. During the surgical procedures, the devices were able to be filled with approximately 0.6 mL of fat.

An animal model study was conducted. A summary of the animal model study is provided in Table 2.

TABLE 2
Animal models.

| | A-0 | A-1 | A-2 | A-3 | A-4 |
|---|---|---|---|---|---|
| DOB | unknown | Dec. 23, | Dec. 24, | Unknown | Unknown |
| | | 2022 | 2022 | | |
| Weight at | 283 | 259 | 266 | 259 | 265 |
| Surgery (g) | | | | | |
| Surgery | 22 May | 25 May | 26 May | 1 Jun. | 1 Jun. |
| Date | 2023 | 2023 | 2023 | 2023 | 2023 |
| Incision | Inferior to | Inferior to | Supra- | Supra- | Supra- |
| Site | the supra- | the supra- | scapular | scapular | scapular |
| | scapular | scapular | area | area | area |
| | area | area | | | |
| Group | PLGA ratio | PLGA ratio | PLGA ratio | PLGA ratio | PLGA ratio |
| | 85:15 | 85:15 | 85:15 | 85:15 | 85:15 |
| Right Pocket | Fat-Filled | Empty | Empty | Empty | Empty |
| | Construct | Construct | Construct | Construct | Construct |
| Left Pocket | Empty | Fat-Filled | Fat-filled | Fat-Filled | Fat-Filled |
| | Construct | Construct | construct | Construct | Construct |
| Fat Injected | 0.6 | 0.6 | 0.6 | 0.52 | 0.4 |
| (ml) | | | | | |
| Construct | Yes | Yes | Yes | No | No |
| Filling | | | | | |
| Intra- | Unexpected | No | Apnea occurred | No | No |
| operative | intraoperative | compli- | post-anesthesia. | compli- | compli- |
| Compli- | death | cations | CPR performed | cations | cations |
| cations | | | successfully. | | |
| Post Op | N/A | Pica: Front | 06/05 - 8 × 3 cm | No | No |
| Compli- | | right paw | fluid collection, | compli- | compli- |
| cations | | biting | enrofloxacin | cations | cations |
| | | | restarted; | | |
| | | | 06/06 - aspirated | | |
| | | | serosanguineous | | |
| | | | fluid, awaiting | | |
| | | | culture results, | | |
| | | | rat stable | | |
| Terminal | N/A | 4 weeks | 2 weeks | 6 weeks | 8 weeks |
| Endpoint | | | | | |

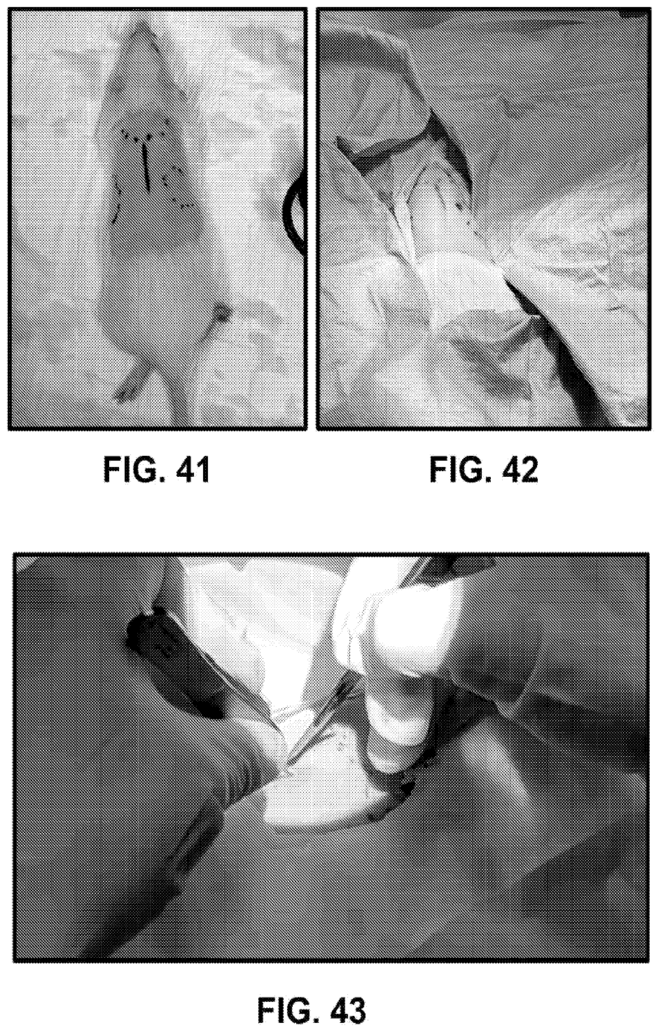

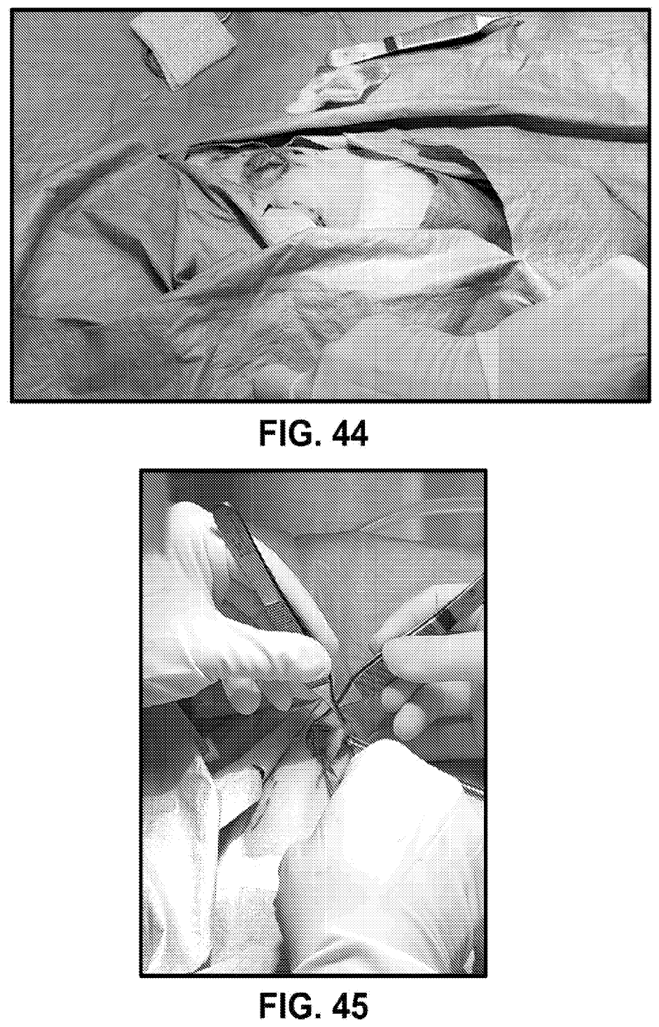

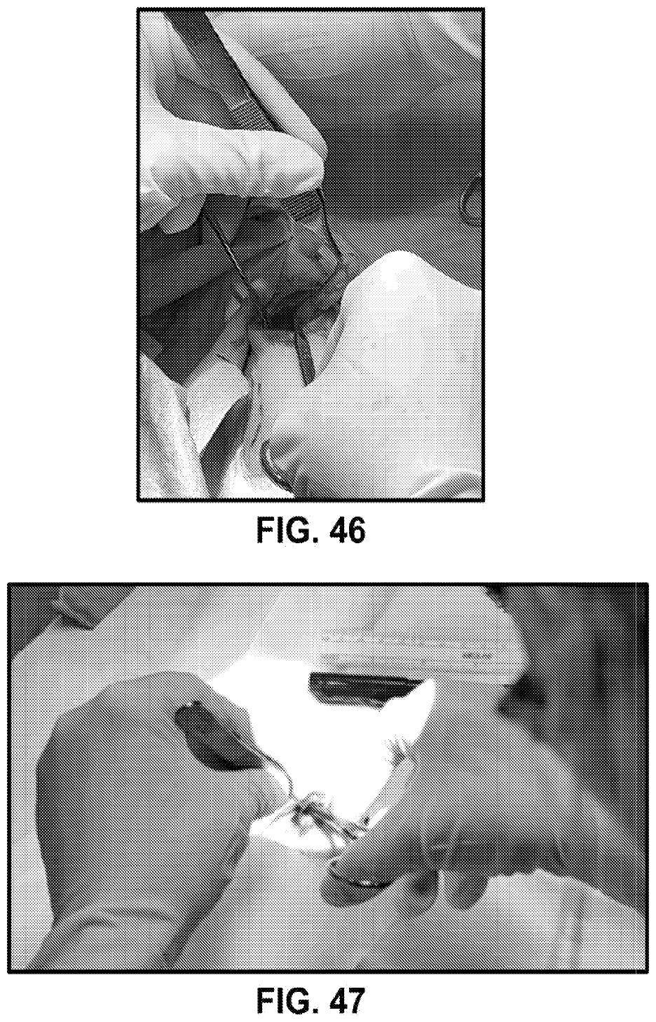

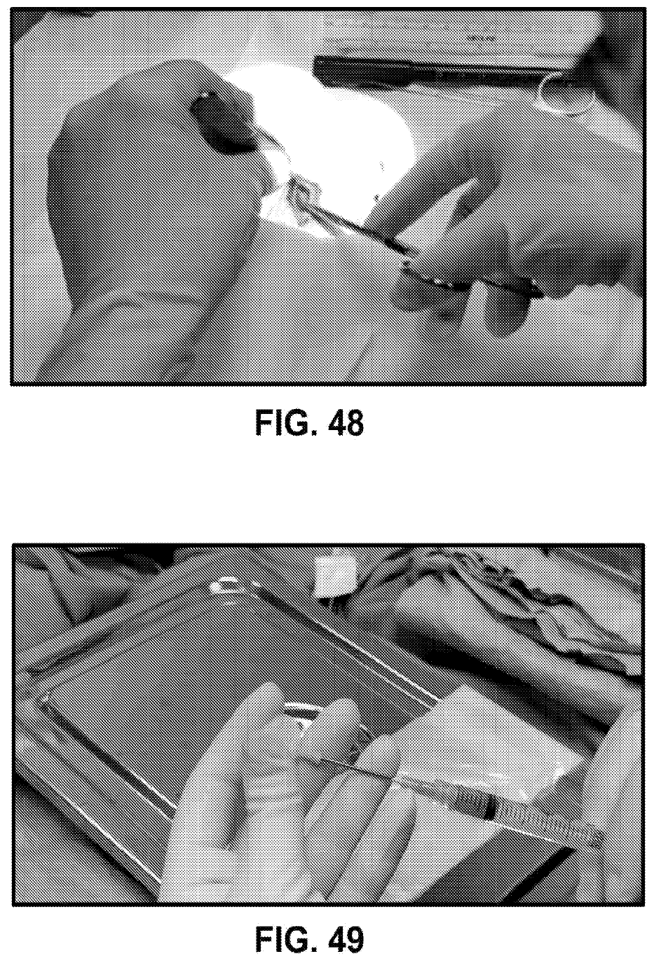

The surgical procedure included anesthesia induction, surgical marking, and incision (FIG. 41-FIG. 44). The surgical procedure further included fat harvesting (FIG. 45-FIG. 46). The surgical procedure further included pocket dissection (FIG. 47-FIG. 48).

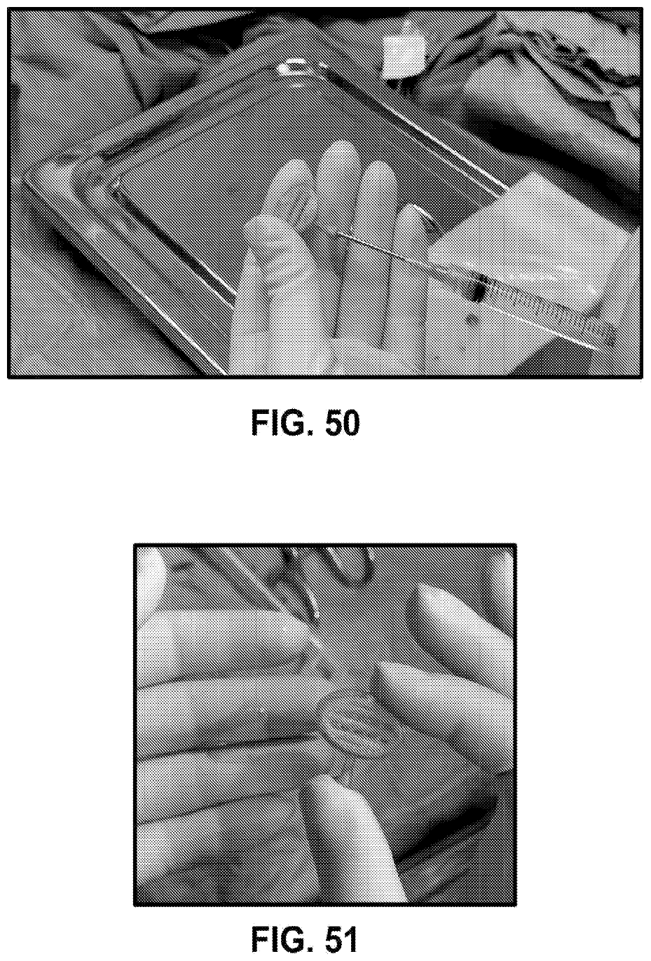

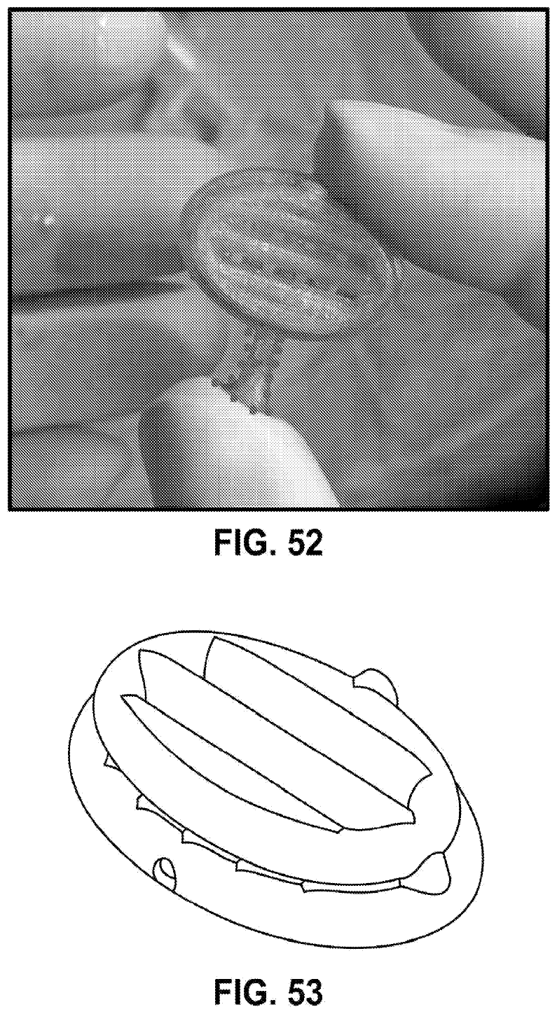

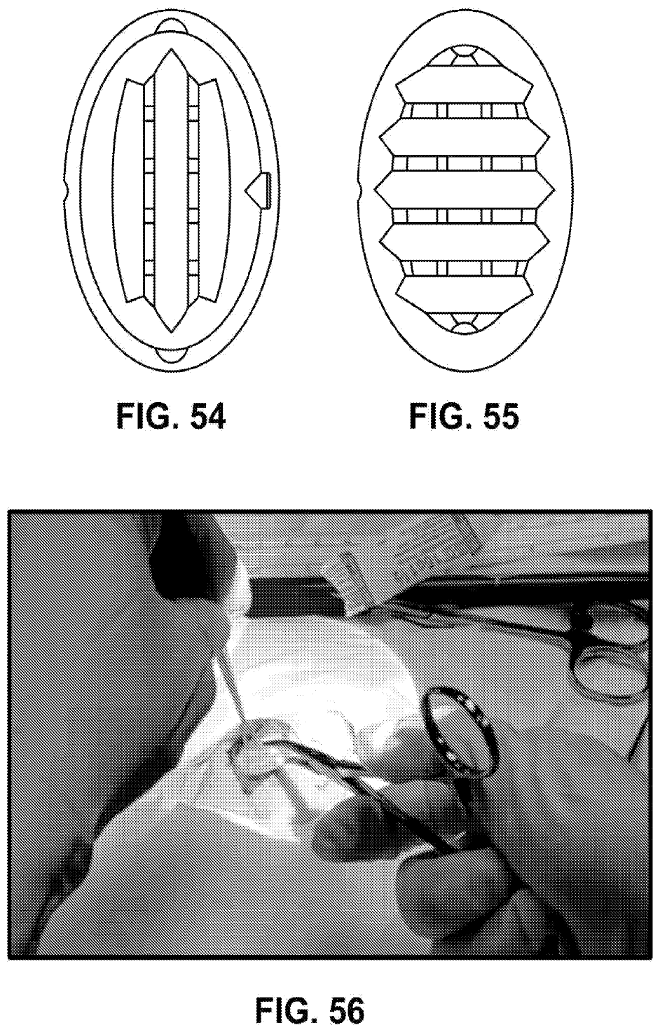

The surgical procedure further included fat processing and construct filling. The harvested fat was washed with 0.9% NaCl. Fat emulsification was achieved by passing it through a 3-way stopcock. Photographs of filling the device with the processed fat are shown in FIG. 49-FIG. 51. After filling, the device inlet was removed by hand (removal area indicated by dotted outline in FIG. 52). Schematic diagrams of the device after inlet removal are shown in FIG. 53-FIG. 55)

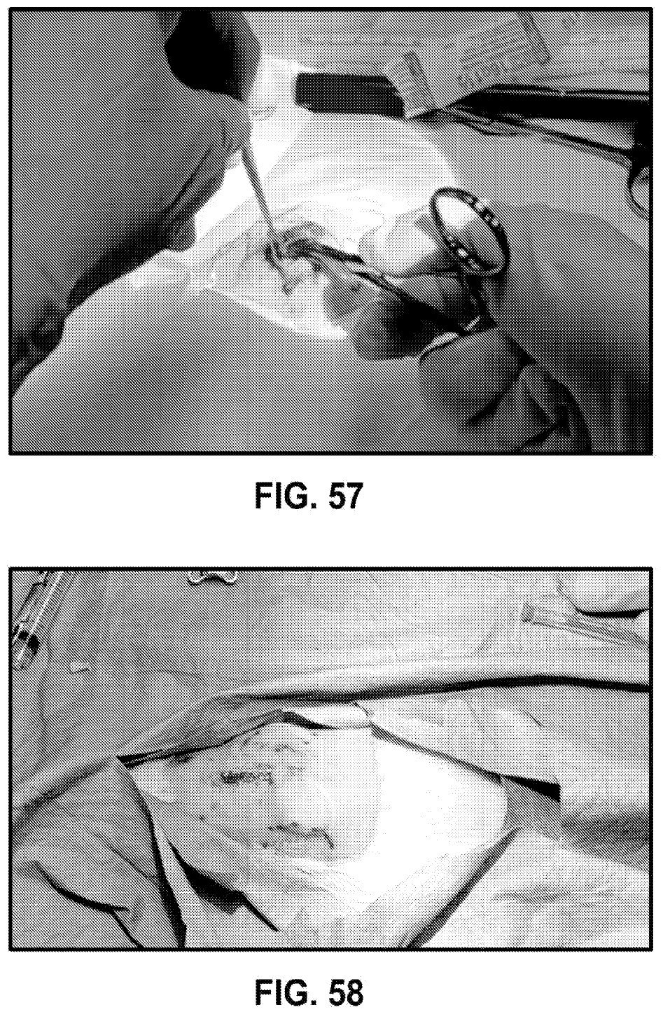

The surgical procedure further included device placement (FIG. 56-FIG. 57), which was secured to the subjacent muscle and fascia using Prolene 5-0. Finally, surgical would closure was performed with surgical staples (FIG. 58).

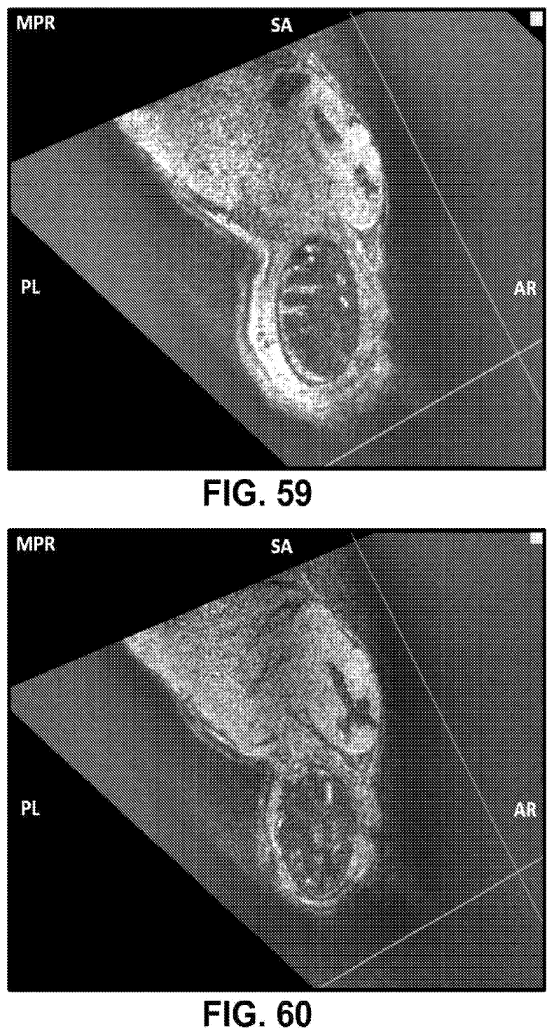

In order to test the feasibility of the imaging procedure, MRI 7T was performed on a rat cadaver with the implanted device (FIG. 59-FIG. 60).

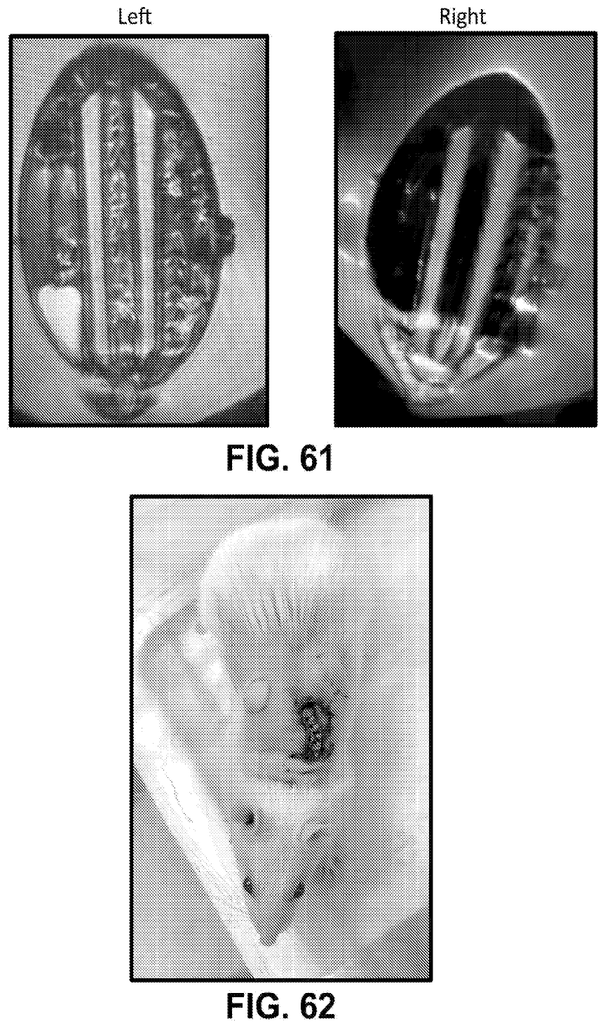

MRI 9.4T was performed on an empty explanted device and one filled with fat (FIG. 61, left is filled with fat, right is empty). The devices were embedded in an agarose gel phantom for proper visualization.

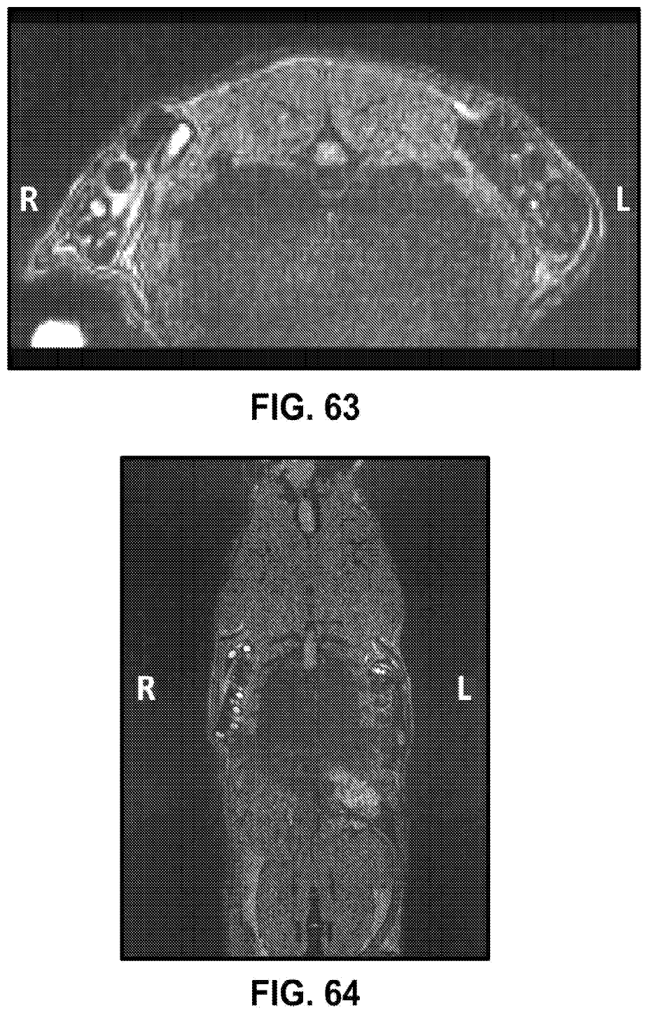

A photograph of rat A-1 post surgery is shown in FIG. 62. 7T MRI images of rat A-1 2-weeks post-surgery are shown in FIG. 63-FIG. 64, where the left device is fat filled and the right device is empty.

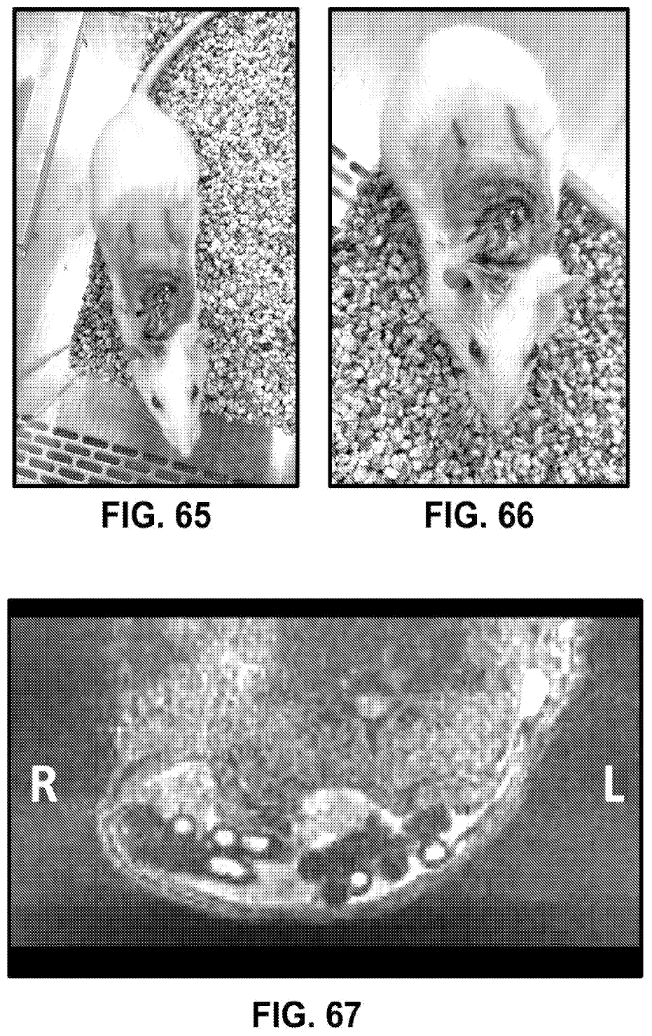

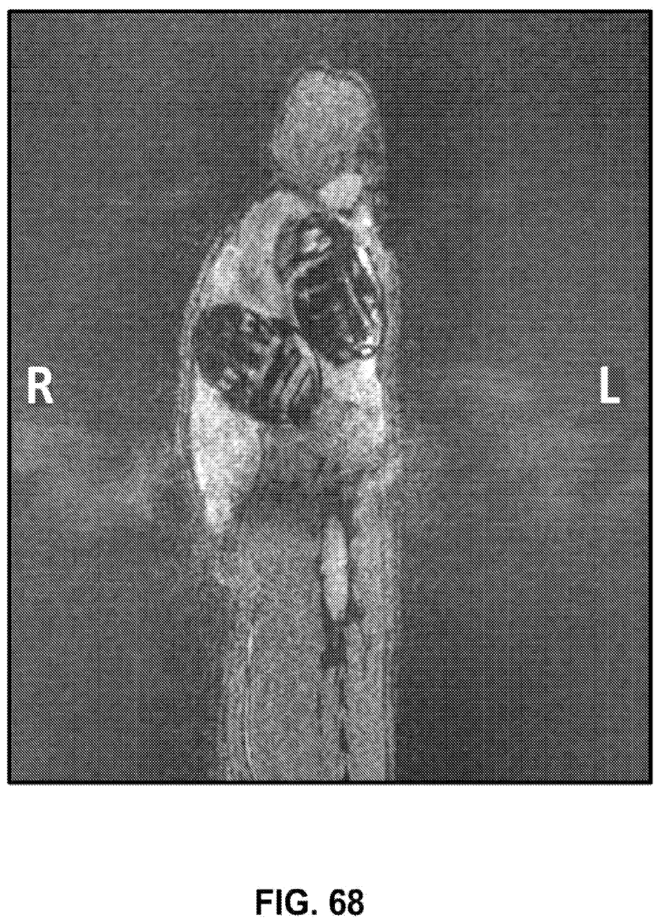

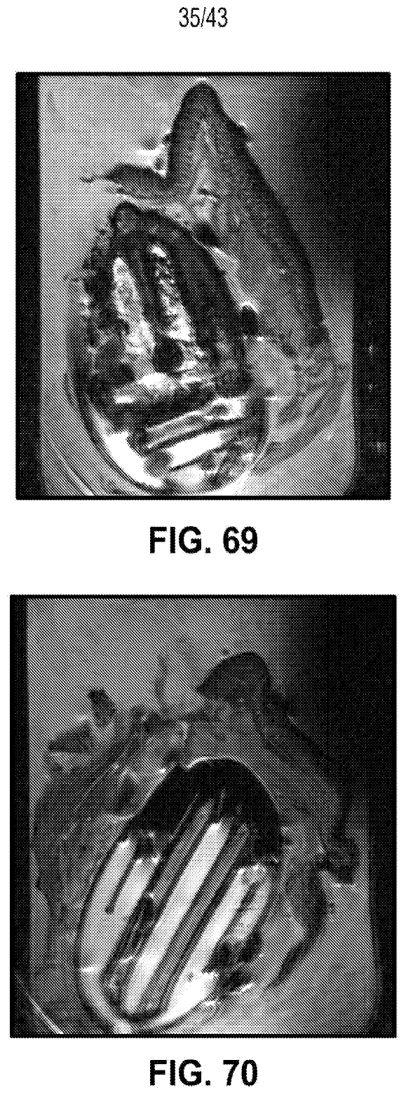

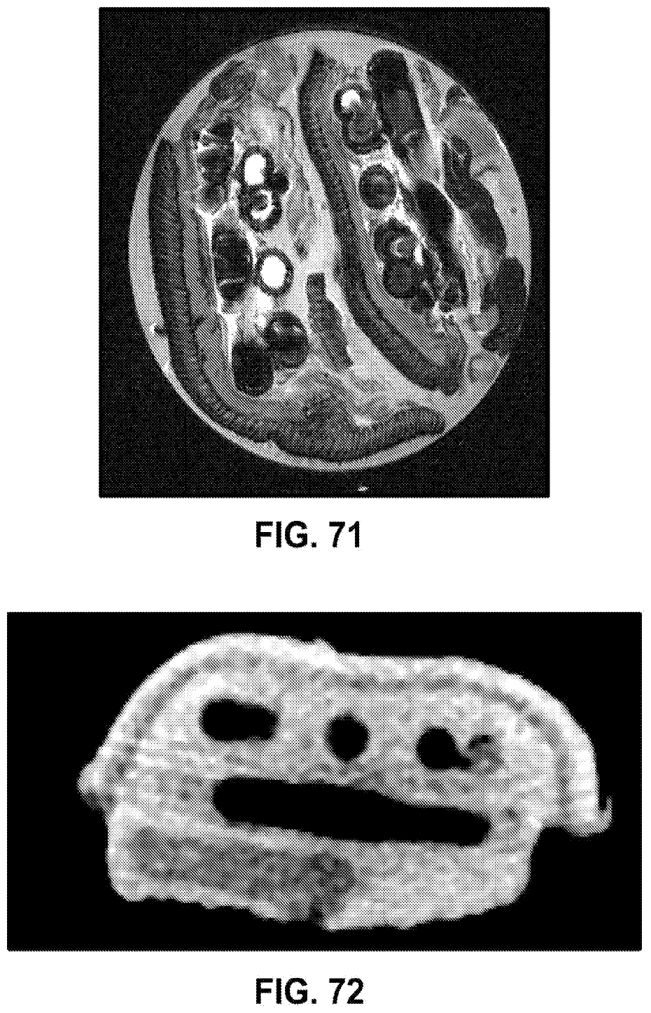

Photographs of rat A-2 post surgery are shown in FIG. 65-FIG. 66. 7T MRI images of rat A-2 2-weeks post-surgery (endpoint for rat A-2) are shown in FIG. 67-FIG. 68, where the left device is fat filled and the right device is empty. 9.4T MRI images of the excised devises of rat A-2 are shown in FIG. 69-FIG. 71.

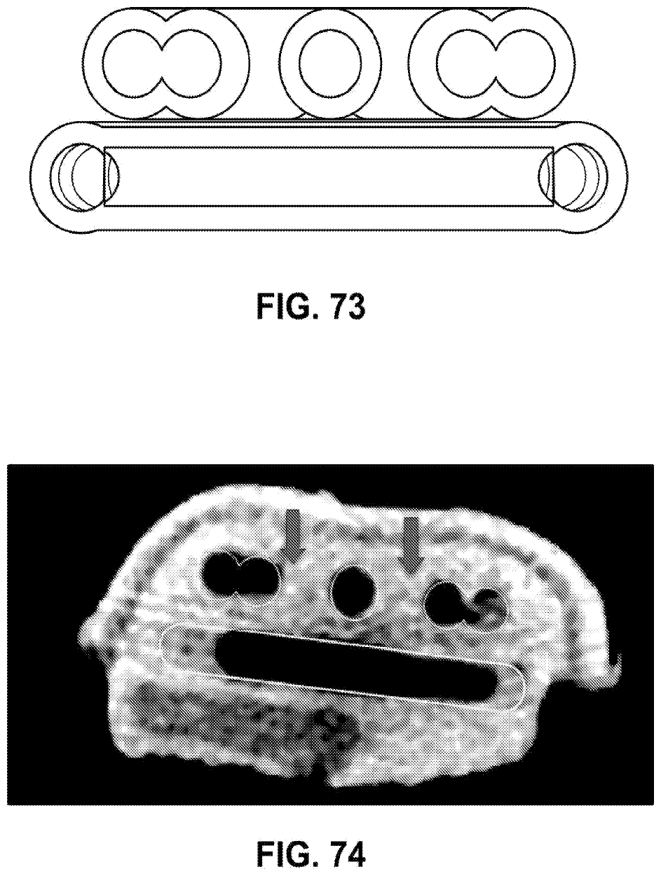

MicroCT angiography of the device from rat A-2 at 2-weeks post-surgery (endpoint) is shown in FIG. 72. PLGA is radio-opaque and the device walls are visible at the microCT. Comparing the microCT image from rat A-2 (FIG. 72) after implantation with the microCT-image of the device before implantation (FIG. 38, FIG. 73), certain differences are observed as illustrated in FIG. 74. The arrows in FIG. 74 could be a fibrotic capsule.

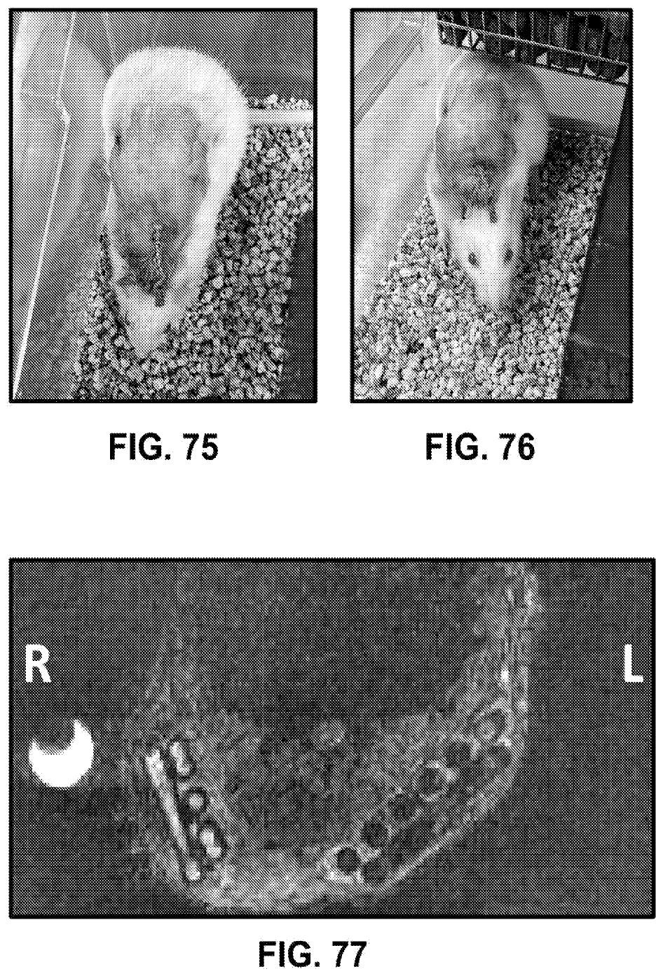

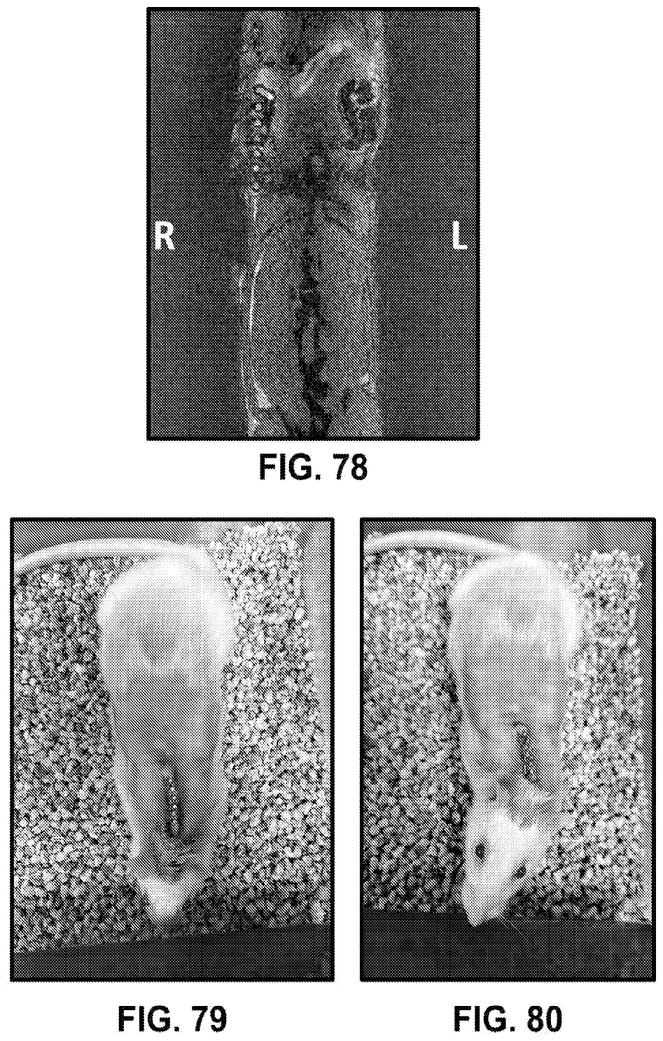

Photographs of rat A-3 post surgery are shown in FIG. 75-FIG. 76. 7T MRI images of rat A-3 two weeks post-surgery are shown in FIG. 77-FIG. 78, where the left device is fat filled and the right device is empty.

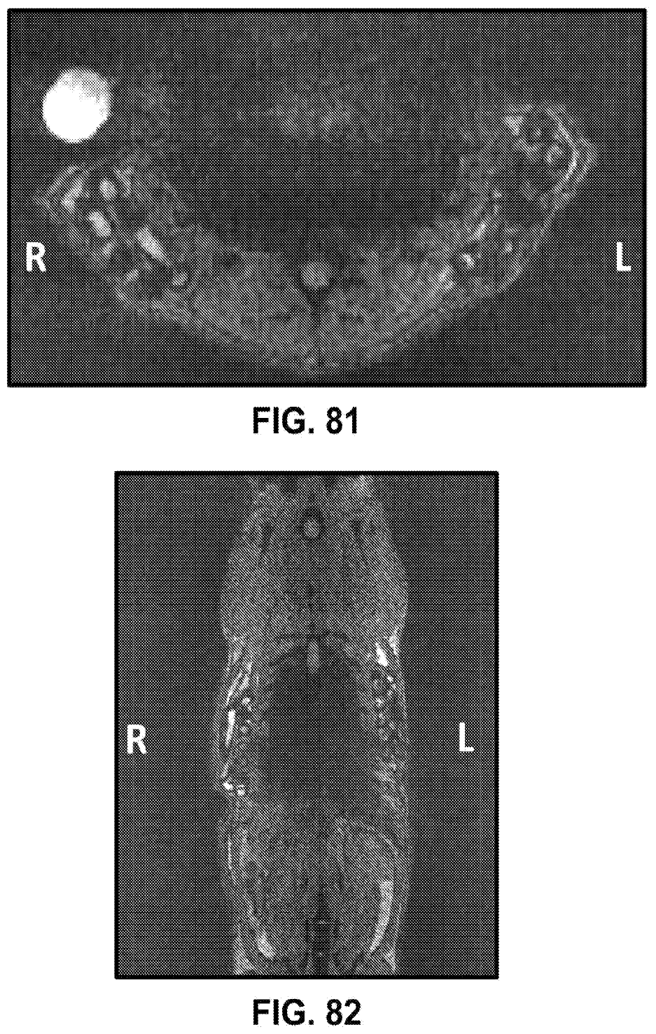

Photographs of rat A-4 post surgery are shown in FIG. 79-FIG. 80. 7T MRI images of rat A-3 two weeks post-surgery are shown in FIG. 81-FIG. 82, where the left device is fat filled and the right device is empty.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

What is claimed is:

1. A device configured to be inserted into an anatomical location of a subject, the device comprising:
a bioresorbable polymer;
wherein, when the device is inserted into the anatomical location of the subject, then device becomes encapsulated by vascular tissue and the bioresorbable polymer is resorbed over an amount of time, thereby forming a chamber in place of the bioresorbable polymer;
wherein the chamber is defined by a boundary formed from the vascular tissue;
wherein the chamber is perfusable; and
wherein the chamber is configured to be at least partially filled with a first plurality of cells.

2. The device of claim 1, wherein the device, before the bioresorbable polymer is resorbed, further includes a compartment defined by the bioresorbable polymer, wherein the compartment is configured to be at least partially filled with a second plurality of cells before or shortly after being inserted into the anatomical location of the subject.

3. The device of claim 2, wherein the compartment is at least partially filled with the second plurality of cells before the bioresorbable polymer is resorbed (e.g., before or shortly after being inserted into the anatomical location of the subject).

4. The device of any of claims 1-3, wherein the compartment is at least partially filled with the second plurality of cells after the bioresorbable polymer is resorbed.

5. The device of any one of claims 1-4, wherein the chamber is at least partially filled with the first plurality of cells after the bioresorbable polymer is resorbed.

6. The device of any one of claims 1-5, wherein the first plurality of cells and/or the second plurality of cells independently comprise adipose tissue.

7. A device configured to be inserted into an anatomical location of a subject, the device comprising:
a plug comprising a bioresorbable polymer; and
a plurality of conduits comprising the bioresorbable polymer;
wherein, when the device is inserted into the anatomical location of the subject, then device becomes encapsulated by vascular tissue and the bioresorbable polymer is resorbed over an amount of time, thereby forming a plurality of channels in place of the plurality of conduits and a port in place of the plug;
wherein each channel is defined by a boundary formed from the vascular tissue;
wherein the port is further defined by the vascular tissue;
wherein the port and the plurality of channels are perfusable;
wherein each of the plurality of channels is fluidly connected to the port; and
wherein the port is configured to receive adipose tissue and the plurality of channels are configured to be at least partially filled with adipose tissue delivered through the port.

8. The device of claim 7, wherein the plurality of conduits are solid, such that the boundary defining each channel is an outer wall.

9. The device of claim 7, wherein the plurality of conduits are hollow, such that the plurality of conduits comprise a wall defining a lumen, the wall comprising the bioresorbable polymer, and each channel is defined by an inner wall and an outer wall, the inner wall and the outer wall being vascular tissue.

10. The device of claim 7, wherein the plurality of conduits comprises:
a first population of conduits; and
a second population of conduits;
each of the first population of conduits and the second population of conduits including one or more of the plurality of conduits;
such that the plurality of channels comprises:
a first population of channels; and
a second population of channels;
each of the first population of channels and the second population of channels including one or more of the plurality of channels;
wherein the first population of conduits are solid, such that boundary defining each of the first population of channels is an outer wall; and
wherein the second population of conduits are hollow such that the each of the second population of conduits comprises a wall defining a lumen, the wall comprising the bioresorbable polymer, and each of the second population of channels is defined by an inner wall and an outer wall, the inner wall and the outer wall being vascular tissue.

11. The device of any one of claims 7-10, wherein each of the plurality of channels, the first population of channels, the second population of channels, or a combination thereof is at least partially filled with adipose tissue after the bioresorbable polymer is resorbed.

12. A device configured to be inserted into an anatomical location of a subject, the device comprising:
a port defined by a boundary comprising a bioresorbable polymer; and
a first plurality of conduits, wherein each of the first plurality of conduits comprises a wall defining a lumen, the wall comprising the bioresorbable polymer;
wherein the port and the lumen of each of the first plurality of conduits is perfusable;
wherein the lumen of each of the first plurality of conduits is fluidly connected to the port;
such that the port is configured to receive a first portion of adipose tissue and the lumen of each of the first plurality of conduits is configured to be at least partially filled with the first portion of adipose tissue delivered through the port;
wherein the device is configured to be at least partially filled with the first portion of adipose tissue before or shortly after being inserted into the anatomical location of the subject (e.g., before the bioresorbable polymer is resorbed);
wherein, when the device is inserted into the anatomical location of the subject, then device becomes encapsulated by vascular tissue and the bioresorbable polymer is resorbed, thereby forming a first plurality of channels in place of the first plurality of conduits, each of the first plurality of channels being defined by a boundary formed from the vascular tissue and each of the first plurality of channels being at least partially filled with the first portion of adipose tissue.

13. The device of claim 12, wherein the device, after the bioresorbable polymer is resorbed, is further configured to receive a second portion of adipose tissue delivered through the port to the first plurality of channels, such that the first plurality of channels are further configured be at least partially filled with a second portion of adipose tissue.

14. The device of claim 12 or claim 13, wherein the device further comprises:
a second plurality of conduits comprising a second bioresorbable polymer, the second plurality of conduits being solid;
such that, when the device is inserted into the anatomical location of the subject, then the second plurality of conduits becomes encapsulated by vascular tissue and the second bioresorbable polymer is resorbed over a second amount of time, thereby forming a second plurality of channels in place of the second plurality of conduits;
wherein each of the second plurality of channels is defined by a boundary formed from the vascular tissue;
wherein the second plurality of channels are perfusable; and
wherein each of the second plurality of channels are fluidly connected to the port;
such that the port is configured to receive a third portion of adipose tissue, and second the plurality of channels are configured to be at least partially filled with the third portion of adipose tissue delivered through the port.

15. The device of claim 14, wherein the second bioresorbable polymer is the same as or different than the bioresorbable polymer.

16. The device of claim 14 or claim 15, wherein the second amount of time is the same as or different than the amount of time.

17. The device of any one of claims 14-16, wherein the third portion of adipose tissue is the same as or different than second portion of adipose tissue.

18. The device of any one of claims 7-17, wherein each of the plurality of conduits, each of the first plurality of conduits, each of the second plurality of conduits, or a combination thereof independently extends from a proximal end to a distal end opposite and spaced apart from the proximal end along a longitudinal axis.

19. The device of any one of claims 7-18, wherein each of the plurality of conduits, each of the first plurality of conduits, each of the second plurality of conduits, or a combination thereof independently further comprises one or more lobes branching off of the longitudinal axis.

20. The device of any one of claims 7-19, wherein each of the plurality of conduits each of the first plurality of conduits, each of the second plurality of conduits, or a combination thereof independently has a cross-sectional shape that is substantially circular.

21. The device of any one of claims 1-21, wherein the chamber, the compartment, each of the plurality of conduits, each of the first plurality of conduits, each of the second plurality of conduits, or a combination thereof independently is further lined with a third plurality of cells.

22. The device of claim 22, wherein the third plurality of cells comprises endothelial cells.

23. The device of any one of claims 1-22, wherein device is formed from a model based on a tessellation of polyhedrons.

24. The device of any one of claims 1-23, wherein the device is formed from a computational 3D space-filling model.

25. The device of any one of claims 1-24, wherein the device has a three-dimensional parametric teardrop shape.

26. The device of any one of claims 1-25, wherein the device has a three-dimensional parametric teardrop shape following the Fibonacci equation.

27. The device of any one of claims 1-26, wherein the device further comprises a therapeutic agent dispersed within the bioresorbable polymer and/or the second bioresorbable polymer.

28. The device of claim 27, wherein the therapeutic agent is dispersed substantially homogeneously throughout the bioresorbable polymer and/or the second bioresorbable polymer.

29. The device of claim 27 or claim 28, wherein the therapeutic agent comprises an anticancer agent, anti-inflammatory agent, analgesic agent, antimicrobial agent, or a combination thereof.

30. The device of any one of claims 27-29, wherein the therapeutic agent comprises a chemotherapeutic agent, an immunotherapeutic agent, or a combination thereof.

31. The device of any one of claims 1-30, wherein the device is anatomically designed for the subject.

32. The device of any one of claims 1-31, wherein the adipose tissue comprises autologous adipose tissue.

33. The device of any one of claims 1-32, wherein the bioresorbable polymer and/or the second bioresorbable polymer is configured to be stable for an amount of time of from 2 weeks to 12 weeks after the device is implanted in the subject.

34. The device of any one of claims 1-33, wherein the bioresorbable polymer and/or the second bioresorbable polymer is porous.

35. The device of any one of claims 1-34, wherein the bioresorbable polymer and/or the second bioresorbable polymer comprises poly(ethylene glycol) diacrylate (PEGDA), poly(ethylene glycol) dimethacrylate (PEGDMA), poly(ethylene glycol) diacrylamide (PEGDAAm), gelatin methacrylate (GelMA), collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, Chitosan methacrylate, polyethylene glycol norbornene, polyethylene glycol dithiol, thiolated gelatin, thiolated chitosan, thiolated silk, PEG based peptide conjugates, cell-adhesive poly(ethylene glycol), MMP-sensitive poly(ethylene glycol), PEGylated fibrinogen, or a combination thereof.

36. The device of any one of claims 1-35, wherein the bioresorbable polymer and/or the second bioresorbable polymer comprises a polyester, such as poly(glycerol-dodecanoate) (PGD).

37. The device of any one of claims 1-36, wherein the bioresorbable polymer and/or the second bioresorbable polymer comprises a poly(ether-ester).

38. The device of any one of claims 1-37, wherein the bioresorbable polymer and/or the second bioresorbable polymer comprises polydioxanone (PDO).

39. The device of any one of claims 1-38, wherein the bioresorbable polymer and/or the second bioresorbable polymer comprises a polyolefin, such as polypropylene.

40. The device of any one of claims 1-39, wherein the bioresorbable polymer and/or the second bioresorbable polymer comprises polyglycolic acid (PGA).

41. The device of any one of claims 1-40, wherein the bioresorbable polymer and/or the second bioresorbable polymer (when present) can each independently comprise polyglycolic acid (PGA) or a copolymer thereof, polylactic acid (PLA) or a copolymer thereof, polycaprolactone (PCL) or a copolymer thereof, or a combination thereof.

42. The device of any one of claims 1-41, wherein the bioresorbable polymer and/or the second bioresorbable polymer comprises a bioink.

43. The device of any one of claims 1-42, wherein the device is produced by additive manufacturing (e.g., 3D printing).

44. The device of any one of claims 1-43, wherein the anatomical location comprises at least a portion of a breast of the subject.

45. The device of any one of claims 1-44, wherein the anatomical location comprises a breast of the subject.

46. A method of manufacturing the device of any one of claims 1-45, the method comprising making the device using additive manufacturing (e.g., 3D printing).

47. The method of claim 46, wherein the method comprises making the device based on a 3D model.

48. The method of claim 47, wherein the 3D model is based on the Fibonacci equation.

49. The method of claim 47 or claim 48, wherein the 3D model is based on an anatomical image of a subject.

50. The method of claim 49, wherein the method further comprises collecting the anatomical image of the subject.

51. A method of treating a subject in need thereof, the method comprising implanting the device of any one of claims 1-45 into the subject.

52. The method of claim 51, wherein the device is at least partially filled with adipose tissue before or after implantation.

53. The method of claim 52, wherein the adipose tissue is autologous adipose tissue.

54. The method of any one of claims 51-53, wherein the device is implanted into a least a portion of a breast of the subject.

55. The method of any one of claims 51-54, wherein the device is implanted into a breast of the subject.

56. The device of any one of claims 51-55, wherein the method comprises full or partial breast reconstruction or augmentation, for example after a lumpectomy or mastectomy, restoring breast volume after a lumpectomy or mastectomy, correcting breast asymmetry, enhancing breast shape or contour, providing support or reinforcement to the breast tissue, improving breast aesthetics, or a combination thereof.

57. The method of any one of claims 51-56, wherein the method comprises breast reconstruction or augmentation.

58. The method of any one of claims 51-57, wherein the method further comprises anatomically designing the device for the subject.
